SEQUENCE LISTING (59 sequences)

SEQ ID NO: 1 - protein, 16 aa, Artificial Sequence
  VL CDR1
KSSQSLLDSDGKTYLN

SEQ ID NO: 2 - protein, 7 aa, Artificial Sequence
  VL CDR2
LVSKLDS

SEQ ID NO: 3 - protein, 9 aa, Artificial Sequence
  VL CDR3
WQGTHFPQT

SEQ ID NO: 4 - protein, 5 aa, Artificial Sequence
  VH CDR1
DYYMH

SEQ ID NO: 5 - protein, 17 aa, Artificial Sequence
  VH CDR2
YIDPENGDTEYAPKFQG

SEQ ID NO: 6 - protein, 5 aa, Artificial Sequence
  VH CDR3
SPFTY

SEQ ID NO: 7 - protein, 23 aa, Artificial Sequence
  VL FR1
DVVMTQSPLSLPVTLGQPASISC

SEQ ID NO: 8 - protein, 15 aa, Artificial Sequence
  VL FR2
WLQQRPGQSPRRLIY

SEQ ID NO: 9 - protein, 32 aa, Artificial Sequence
  VL FR3
GVPDRFSGSGSGTDFTLKISRVEAEDVGVYYC

SEQ ID NO: 10 - protein, 10 aa, Artificial Sequence
  VL FR4
FGGGTKVEIK

SEQ ID NO: 11 - protein, 30 aa, Artificial Sequence
  VH FR1
QVQLVQSGAEVKKPGASVKVSCKASGFNIK

SEQ ID NO: 12 - protein, 14 aa, Artificial Sequence
  VH FR2
WVRQAPGQGLEWIG

SEQ ID NO: 13 - protein, 32 aa, Artificial Sequence
  VH FR3
RATMTADTSISTAYMELSRLRSDDTAVYYCRS

SEQ ID NO: 14 - protein, 11 aa, Artificial Sequence
  VH FR4
WGQGTLVTVSS

SEQ ID NO: 15 - protein, 444 aa, Artificial Sequence
  ch7D1 heavy chain amino acid
EVKLVESGAELVRSGASVKLSCTASGFNIKDYYMHWVKQRPEQGLEWIGYIDPENGDTEYAPKFQGKATMTADTSSNTAYLQLSSLTSEDTAVYYCRSSPFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1332 bp, Artificial Sequence
  ch7D1 heavy chain DNA
gaggtgaagctggtggagagcggagcagagctggtgcggtccggagcctctgtgaagctgagctgcacagcctccggcttcaacatcaaggattactatatgcactgggtgaagcagcggcccgagcagggcctggagtggatcggctacatcgaccccgagaacggcgataccgagtatgcccctaagtttcagggcaaggccaccatgacagccgacaccagctccaatacagcctacctccagctgtctagcctgaccagcgaggatacagccgtgtactattgcaggtcctctcccttcacctattggggacagggcaccctggtgacagtgagcgccgcctccacaaaaggcccctccgtgtttccactggcccccagctccaagagcacctccggaggcacagccgccctgggctgtctggtgaaggactacttcccagagcccgtgaccgtgtcttggaacagcggcgccctgaccagcggagtgcacacatttcccgccgtgctccagagcagcggcctgtactccctgtcctctgtggtgaccgtgccaagctcctctctgggcacccagacatatatctgcaacgtgaatcacaagcctagcaatacaaaggtggacaagaaggtggagccaaagtcctgtgataagacccacacatgccccccttgtcctgcaccagagctgctgggcggcccaagcgtgttcctgtttccacccaagcccaaggacaccctgatgatctctcgcaccccagaggtgacatgcgtggtggtggacgtgagccacgaggaccccgaggtgaagttcaactggtacgtggatggcgtggaggtgcacaatgccaagaccaagcctcgggaggagcagtacaattctacctatagagtggtgagcgtgctgacagtgctgcaccaggattggctgaacggcaaggagtataagtgcaaggtgtccaataaggccctgcccgcccctatcgagaaaaccatcagcaaggccaagggccagcctagggagccacaggtgtacacactgcctccatcccgcgaggagatgaccaagaaccaggtgtctctgacatgtctggtgaagggcttctatcccagcgacatcgccgtggagtgggagtccaatggccagcctgagaacaattacaagaccacaccccctgtgctggactccgatggctctttctttctgtattccaagctgaccgtggataagtctcggtggcagcagggcaacgtgttttcttgtagcgtgatgcacgaggccctgcacaatcactacacacagaagtccctgtctctgagccccggcaag

SEQ ID NO: 17 - protein, 219 aa, Artificial Sequence
  ch7D1 light chain amino acid
DVLMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - DNA, 657 bp, Artificial Sequence
  ch7D1 light chain DNA
gatgtgctgatgacccagacacctctgaccctgtccgtgacaatcggccagccagccagcatctcctgcaagagcagccagtccctgctggactctgatggcaagacatacctgaactggctgctccagcggcccggccagagccccaagagactgatctatctggtgagcaagctggactccggcgtgcccgataggttcaccggctctggcagcggcaccgacttcaccctgaagatcagccgggtggaggcagaggatctgggcgtgtactattgctggcagggcacccacttcccccagacatttggcggaggcaccaagctggagctgaagaggacagtggcagcaccttccgtgttcatctttcccccttctgacgagcagctgaagtctggcaccgccagcgtggtgtgcctgctgaacaatttctacccaagggaggccaaggtgcagtggaaggtggataacgccctccagtccggcaattctcaggagagcgtgacagagcaggactccaaggattctacctatagcctgtctagcaccctgacactgtctaaggccgactacgagaagcacaaggtgtatgcctgcgaggtgacacaccagggcctgtcctctcccgtgaccaagtcctttaatcggggcgagtgt

SEQ ID NO: 19 - protein, 444 aa, Artificial Sequence
  hz7D1.11 heavy chain amino acid
QVQLVQSGAEVKKPGASVKVSCKASGFNIKDYYMHWVRQAPGQGLEWIGYIDPENGDTEYAPKFQGRATMTADTSISTAYMELSRLRSDDTAVYYCRSSPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1332 bp, Artificial Sequence
  hz7D1.11 heavy chain DNA
caggtgcagctggtgcagtccggagcagaggtgaagaagccaggcgcctctgtgaaggtgagctgcaaggcctccggcttcaacatcaaggactactatatgcactgggtgaggcaggcccctggccagggcctggagtggatcggctacatcgaccccgagaatggcgataccgagtatgcccctaagtttcagggccgcgccaccatgacagccgatacctccatctctacagcctacatggagctgtctcggctgagaagcgacgataccgccgtgtactactgccggagcagcccattcacatattggggccagggcaccctggtgacagtgtctagcgcctccaccaaaggccccagcgtgttcccactggccccctcctctaagagcacctccggaggcacagccgccctgggctgtctggtgaaggattacttcccagagcccgtgacagtgtcctggaactctggcgccctgacctccggagtgcacacatttcccgccgtgctccagagcagcggcctgtacagcctgtctagcgtggtgaccgtgccttcctctagcctgggcacccagacatatatctgcaacgtgaatcacaagccttccaatacaaaggtggacaagaaggtggagccaaagtcttgtgataagacccacacatgccccccttgtcctgcaccagagctgctgggcggcccaagcgtgttcctgtttccacccaagcccaaggacaccctgatgatctcccggaccccagaggtgacatgcgtggtggtggacgtgagccacgaggaccccgaggtgaagttcaactggtacgtggatggcgtggaggtgcacaatgccaagaccaagcctagggaggagcagtacaacagcacctatcgcgtggtgtccgtgctgacagtgctgcaccaggactggctgaacggcaaggagtataagtgcaaggtgtccaataaggccctgcccgcccctatcgagaaaaccatcagcaaggcaaagggacagcctcgggagccacaggtgtacacactgcctcccagccgggaggagatgaccaagaaccaggtgagcctgacatgtctggtgaagggcttctatccctccgacatcgccgtggagtgggagtctaatggccagcctgagaacaattacaagaccacaccccctgtgctggacagcgatggctccttctttctgtattctaagctgaccgtggataagagcagatggcagcagggcaacgtgttttcttgtagcgtgatgcacgaggccctgcacaatcactacacacagaagtccctgtctctgagccccggcaag

SEQ ID NO: 21 - protein, 219 aa, Artificial Sequence
  hz7D1.11 light chain amino acid
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 657 bp, Artificial Sequence
  hz7D1.11 light chain DNA
gacgtggtcatgacccagtctccactgagcctgcccgtgacactgggacagccagccagcatctcctgcaagagcagccagtccctgctggactctgatggcaagacctacctgaactggctccagcagaggcctggccagtctccacggagactgatctatctggtgagcaagctggactccggcgtgcccgataggttctctggcagcggctccggcaccgactttacactgaagatcagccgcgtggaggcagaggatgtgggcgtgtactattgctggcagggcacccacttcccccagacatttggcggcggcaccaaggtggagatcaagcggacagtggccgccccttccgtgttcatctttcccccttctgacgagcagctgaagtctggcaccgccagcgtggtgtgcctgctgaacaatttctaccctagagaggccaaggtgcagtggaaggtggataacgccctccagtccggcaattctcaggagagcgtgacagagcaggactccaaggattctacctatagcctgtctagcaccctgacactgagcaaggccgactacgagaagcacaaggtgtatgcctgcgaggtgacacaccagggcctgtcctctccagtgaccaagtcctttaatagaggcgagtgt

SEQ ID NO: 23 - protein, 444 aa, Artificial Sequence
  hz7D1.22 heavy chain amino acid
QVQLVQSGAEVKKPGASVKVSCKASGYTFKDYYMHWVRQAPGQGLEWMGYIDPENGDTEYAPKFQGRVTMTADTSISTAYMELSRLRSDDTAVYYCRSSPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 1332 bp, Artificial Sequence
  hz7D1.22 heavy chain DNA
caggtgcagctggtgcagtccggagcagaggtgaagaagccaggcgcctctgtgaaggtgagctgcaaggcctccggctacacattcaaggactactatatgcactgggtgaggcaggcccctggccagggcctggagtggatgggctacatcgaccccgagaacggcgataccgagtatgcccctaagtttcagggccgcgtgaccatgacagccgatacctccatctctacagcctacatggagctgtctcggctgagaagcgacgataccgccgtgtactactgccggagcagcccattcacatattggggccagggcaccctggtgacagtgtctagcgcctccaccaaaggccccagcgtgttcccactggccccctcctctaagagcacctccggaggcacagccgccctgggctgtctggtgaaggattatttcccagagcccgtgaccgtgtcctggaactctggcgccctgacctccggagtgcacacatttcccgccgtgctccagagcagcggcctgtacagcctgtctagcgtggtgaccgtgccttcctctagcctgggcacccagacatatatctgcaacgtgaatcacaagccttccaatacaaaggtggacaagaaggtggagccaaagtcttgtgataagacccacacatgccccccttgtcctgcaccagagctgctgggcggcccaagcgtgttcctgtttccacccaagcccaaggacaccctgatgatctcccggaccccagaggtgacatgcgtggtggtggacgtgagccacgaggaccccgaggtgaagttcaactggtacgtggatggcgtggaggtgcacaatgccaagaccaagcctagggaggagcagtacaatagcacctatcgcgtggtgtccgtgctgacagtgctgcaccaggactggctgaacggcaaggagtataagtgcaaggtgtccaataaggccctgcccgcccctatcgagaaaaccatcagcaaggcaaagggacagcctcgggagccacaggtgtacacactgcctcccagccgggaggagatgaccaagaaccaggtgagcctgacatgtctggtgaagggcttctatccctccgacatcgccgtggagtgggagtctaatggccagcctgagaacaattacaagaccacaccccctgtgctggacagcgatggctccttctttctgtattctaagctgaccgtggataagagcagatggcagcagggcaacgtgttttcttgtagcgtgatgcacgaggccctgcacaatcactacacacagaagtccctgtctctgagccccggcaag

SEQ ID NO: 25 - protein, 219 aa, Artificial Sequence
  hz7D1.22 light chain amino acid
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGTHFPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 657 bp, Artificial Sequence
  hz7D1.22 light chain DNA
gacgtggtcatgacccagtctccactgagcctgcccgtgacactgggacagccagccagcatctcctgcaagagcagccagtccctgctggactctgatggcaagacctacctgaactggttccagcagaggcctggccagtctccacggagactgatctatctggtgagcaagctggactccggcgtgcccgataggttctctggcagcggctccggcaccgactttacactgaagatcagccgcgtggaggcagaggatgtgggcgtgtactattgctggcagggcacccacttcccccagacatttggcggcggcaccaaggtggagatcaagcggacagtggccgccccttccgtgttcatctttcccccttctgacgagcagctgaagtctggcaccgccagcgtggtgtgcctgctgaacaatttctaccctagagaggccaaggtgcagtggaaggtggataacgccctccagtccggcaattctcaggagagcgtgacagagcaggactccaaggattctacctatagcctgtctagcaccctgacactgagcaaggccgactacgagaagcacaaggtgtatgcctgcgaggtgacacaccagggcctgtcctctccagtgaccaagtcctttaatagaggcgagtgt

SEQ ID NO: 27 - protein, 10 aa, Artificial Sequence
  PCSK9 Ag epitope
PEEDGTRFHR

SEQ ID NO: 28 - protein, 15 aa, Artificial Sequence
  PCSK9 Ag epitope 1
AKDPWRLPGTYVVVL

SEQ ID NO: 29 - protein, 3 aa, Artificial Sequence
  PCSK9 Ag epitope 2
FAQ

SEQ ID NO: 30 - protein, 4 aa, Artificial Sequence
  PCSK9 Ag epitope 3
CQGK

SEQ ID NO: 31 - protein, 2 aa, Artificial Sequence
  PCSK9 Ag epitope 4
NF

SEQ ID NO: 32 - protein, 6 aa, Artificial Sequence
  PCSK9 Ag epitope 5
LGTLGT

SEQ ID NO: 33 - protein, 5 aa, Artificial Sequence
  PCSK9 Ag epitope 6
VSQSG

SEQ ID NO: 34 - protein, 10 aa, Artificial Sequence
  PCSK9 Ag epitope 8
EDQRVLTPNL

SEQ ID NO: 35 - protein, 7 aa, Artificial Sequence
  PCSK9 Ag epitope 9
CRTVWSA

SEQ ID NO: 36 - protein, 4 aa, Artificial Sequence
  PCSK9 Ag epitope 10
CAPD

SEQ ID NO: 37 - protein, 9 aa, Artificial Sequence
  PCSK9 Ag epitope 11
HKPPVLRPR

SEQ ID NO: 38 - protein, 12 aa, Artificial Sequence
  PCSK9 Ag epitope 12
STTGSTSEGAVT

SEQ ID NO: 39 - protein, 113 aa, Artificial Sequence
  IGHV1-2*02/IGHJ4*03
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARYFDYWGQGTLVTVSS

SEQ ID NO: 40 - protein, 112 aa, Artificial Sequence
  IGKV2-30*01/IGKJ4*2
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPLTFGGGTKVEIK

SEQ ID NO: 41 - DNA, 343 bp, Artificial Sequence
  Heavy chain variable region DNA of m7D1 antibody
gaggtgaagctggtggagtctggggcagagcttgtgaggtcaggggcctcagtcaagttgtcctgcacagcttctggcttcaacattaaagactactatatgcactgggtgaagcagaggcctgaacagggcctggagtggattggatatattgatcctgagaatggtgatactgaatatgccccgaagttccagggcaaggccactatgactgcagacacatcctccaacacagcctacctgcagctcagcagcctgacatctgaggacactgccgtctattactgtcgtagtagcccgtttacttactggggccaagggactctggtcactgtctctgcag

SEQ ID NO: 42 - DNA, 337 bp, Artificial Sequence
  Light chain variable region DNA of m7D1 antibody
gatgttttgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggagggaccaagctggagctgaaag

SEQ ID NO: 43 - protein, 114 aa, Artificial Sequence
  Heavy chain variable region of m7D1 antibody
EVKLVESGAELVRSGASVKLSCTASGFNIKDYYMHWVKQRPEQGLEWIGYIDPENGDTEYAPKFQGKATMTADTSSNTAYLQLSSLTSEDTAVYYCRSSPFTYWGQGTLVTVSA

SEQ ID NO: 44 - protein, 112 aa, Artificial Sequence
  Light chain variable region of m7D1 antibody
DVLMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLELK

SEQ ID NO: 45 - protein, 114 aa, Artificial Sequence
  Heavy chain variable region of 7D1 antibody
QVQLVQSGAEVKKPGASVKVSCKASGFNIKDYYMHWVRQAPGQGLEWIGYIDPENGDTEYAPKFQGRATMTADTSISTAYMELSRLRSDDTAVYYCRSSPFTYWGQGTLVTVSS

SEQ ID NO: 46 - protein, 112 aa, Artificial Sequence
  Light chain variable region of 7D1 antibody
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 47 - DNA, 344 bp, Artificial Sequence
  Heavy chain variable region DNA of 7D1 antibody
caggtgcagctggtgcagtccggagcagaggtgaagaagccaggcgcctctgtgaaggtgagctgcaaggcctccggcttcaacatcaaggactactatatgcactgggtgaggcaggcccctggccagggcctggagtggatcggctacatcgaccccgagaatggcgataccgagtatgcccctaagtttcagggccgcgccaccatgacagccgatacctccatctctacagcctacatggagctgtctcggctgagaagcgacgataccgccgtgtactactgccggagcagcccattcacatattggggccagggcaccctggtgacagtgtctagcgc

SEQ ID NO: 48 - DNA, 337 bp, Artificial Sequence
  Light chain variable region DNA of 7D1 antibody
gacgtggtcatgacccagtctccactgagcctgcccgtgacactgggacagccagccagcatctcctgcaagagcagccagtccctgctggactctgatggcaagacctacctgaactggctccagcagaggcctggccagtctccacggagactgatctatctggtgagcaagctggactccggcgtgcccgataggttctctggcagcggctccggcaccgactttacactgaagatcagccgcgtggaggcagaggatgtgggcgtgtactattgctggcagggcacccacttcccccagacatttggcggcggcaccaaggtggagatcaagc

SEQ ID NO: 49 - protein, 693 aa, Artificial Sequence
  PCSK9[homo sapiens]
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQA

SEQ ID NO: 50 - protein, 614 aa, Artificial Sequence
  PCSK9 fragment
MGTVSSRRSWWPLPLLLLLLLLLGPAGARAQEDEDGDYEELVLALRSEEDGLAEAPEHGTTATFHRCKEETHLSQSERTARRLQAQAARRGYLTKILHVFHGLLPGFLVKMSGDLLELALKLPHVDYIEEDSSVSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNGTVSGTLIGLEFIRKSQLVQPVGPLVVLLPLAGGYSRVLNAACQRLARAGVVLVTAAGRDDACLYSPASAPEVITVGATNAQDQPVTNFGRCVDLFAPGIIGASSDCSTCFTSQAAAHVAGIAAMMLSAEPELTLAELRQRLIHFSAKDVINEAWFPVAALPPSTHGAGWQLFHSGPTRMATAVAREELLSCSSFSRSGKRRGERMEAQGGKLVCRAHNAFGGEGVYAIARCCLLPQANCSVHTAPPAEASMGTRVHCHQQGHVLTGCSSHWEVEDLGTGQPNQCVGHREASIHASCCHAPGLECKVKEHGIPAPQEQVTVACEEGWTLTGCSALPGTSHVLGAYAVDNTCVVRSRDVAVAICCRSRHLAQASQELQA

SEQ ID NO: 51 - protein, 16 aa, Artificial Sequence
  LCDR1 of m7D1
KSSQSLLDSDGKTYLN

SEQ ID NO: 52 - protein, 7 aa, Artificial Sequence
  LCDR2 of m7D1
LVSKLDS

SEQ ID NO: 53 - protein, 9 aa, Artificial Sequence
  LCDR3 of m7D1
WQGTHFPQT

SEQ ID NO: 54 - protein, 5 aa, Artificial Sequence
  HCDR1 of m7D1
DYYMH

SEQ ID NO: 55 - protein, 17 aa, Artificial Sequence
  HCDR2 of m7D1
YIDPENGDTEYAPKFQG

SEQ ID NO: 56 - protein, 5 aa, Artificial Sequence
  HCDR3 of m7D1
SPFTY

SEQ ID NO: 57 - protein, 809 aa, Artificial Sequence
  GST-PCSK9 amino acid sequence
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSQEDEDGDYEELVLALRSEEDGLAEAPEHGTTATFHRCKEETHLSQSERTARRLQAQAARRGYLTKILHVFHGLLPGFLVKMSGDLLELALKLPHVDYIEEDSSVSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNGTVSGTLIGLEFIRKSQLVQPVGPLVVLLPLAGGYSRVLNAACQRLARAGVVLVTAAGRDDACLYSPASAPEVITVGATNAQDQPVTNFGRCVDLFAPGIIGASSDCSTCFTSQAAAHVAGIAAMMLSAEPELTLAELRQRLIHFSAKDVINEAWFPVAALPPSTHGAGWQLFHSGPTRMATAIAREELLSCSSFSRSGKRRGERMEAQGGKLVCRAHNAFGGEGVYAIARCCLLPQANCSVHTAPPAEASMGTRVHCHQQGHVLTGCSSHWEVEDLGTGQPNQCVGHREASIHASCCHAPGLECKVKEHGIPAPQEQVTVACEEGWTLTGCSALPGTSHVLGAYAVDNTCVVRSRDVAVAICCRSRHLAQASQELQ

SEQ ID NO: 58 - DNA, 2427 bp, Artificial Sequence
  GST-PCSK9 DNA sequence
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatcccaagaagatgaagatggtgattatgaggaactggtgctggcgctgcgtagcgaagaggacggtctggcggaggcgccggaacacggtaccaccgcgaccttccaccgttgcaaagaggaaacccacctgagccagagcgaacgtaccgcgcgtcgtctgcaagcgcaagcggcgcgtcgtggctacctgaccaaaatcctgcacgtgttccacggtctgctgccgggctttctggttaagatgagcggtgacctgctggagctggcgctgaaactgccgcacgtggactatattgaggaagatagcagcgttagcatcccgtggaacctggagcgtattaccccgccgcgttaccgtgcggacgaatatcagccgccggatggtggtagcctggttgaggtgtacctgctggacaccagcatccaaagcgatcaccgtgagattgaaggtcgtgtgatggttaccgacttcgaaaacgtgccggaggaagatggcacccgttttcaccgtcaggcgagcaaatgcgacagccacggcacccatctggcgggtgtggttagcggccgtgatgcgggtgttgcgaaaggcgcgagcatgcgtagcctgcgtgtgctgaacggcaccgtgagcggcaccctgatcggtctggagttcattcgtaagagccagctggtgcaaccggttggtccgctggttgtgctgctgccgctggcgggtggctacagccgtgtgctgaacgcggcgtgccagcgtctggcgcgtgcgggcgtggttctggttaccgcggcgggtcgtgatgatgcgtgcctgtatagcccggcgagcgcgccggaagtgatcaccgttggtgcgaccaacgcgcaggaccaaccggtgaccaactttggtcgttgcgtggacctgttcgcgccgggtatcattggcgcgagcagcgactgcagcacctgctttaccagccaagctgcggcgcatgttgcgggtattgcggcgatgatgctgagcgcggagccggaactgaccctggcggaactgcgtcaacgtctgatccacttcagcgcgaaagatgtgattaacgaggcgtggtttccggttgcggcgctgccgccgagcacccacggtgcgggttggcagctgtttcatagcggtccgacccgtatggcgaccgcgattgcgcgtgaggaactgctgagctgcagcagctttagccgtagcggcaagcgtcgtggtgagcgtatggaagcgcagggtggcaaactggtgtgccgtgcgcacaacgcgtttggtggcgaaggcgtttacgcgattgcgcgttgctgcctgctgccgcaagcgaactgcagcgtgcacaccgctccgccggcggaggcgagcatgggcacccgtgtgcactgccaccagcaaggccacgttctgaccggttgcagcagccactgggaagtggaagatctgggcaccggccagccgaaccaatgcgttggtcaccgtgaagcgagcattcatgcgagctgctgccatgcgccgggcctggagtgcaaggttaaagaacacggtattccggcgccgcaggagcaagtgaccgttgcgtgcgaggaaggctggaccctgaccggttgcagcgcgctgccgggcaccagccacgtgctgggtgcgtatgcggttgacaacacctgcgtggttcgtagccgtgatgtggcggtggcgatctgctgccgtagccgtcatctggcgcaagcgagccaagaactgcaa

SEQ ID NO: 59 - protein, 2 aa, Artificial Sequence
  PCSK9 Ag epitope 7
ED

(19) United States
(12) Patent Application Publication
(10) Pub. No.: US 2023/0242673 A1
(43) Pub. Date: Aug. 3, 2023

(54) ANTI-PCSK9 ANTIBODY AND USE THEREOF

(71) Applicant: BIOSTREAM TECHNOLOGIES CO., LTD., Yongin-si Gyeonggi-do (KR)

(72) Inventors: Hyunho LEE, Suwon-si, Gyeonggi-do (KR); Joowoong PARK, Seoul (KR); Taewook KANG, Yongin-si Gyeonggi-do (KR)

(21) Appl. No.: 18/003,121

(22) PCT Filed: Jun. 23, 2021
(86) PCT No.: PCT/KR2021/007876

§ 371 Date: Dec. 22, 2022

(30) Foreign Application Priority Data
Jun. 24, 2020 (KR) 10-2020-0077370

(51) Int. Cl. C07K 16/40; A61P 3/06; C12N 15/63

(52) U.S. Cl. CPC C07K 16/40; A61P 3/06; C12N 15/63; C07K 2317/24; C07K 2317/565

(57) ABSTRACT

An anti-PCSK9 antibody of the present invention specifically binds to PCSK9 in spite of binding to an epitope different from that of the conventional anti-PCSK9 antibody. In particular, the anti-PCSK9 antibody of the present invention has very good binding ability with PCSK9. Therefore, the anti-PCSK9 antibody may effectively block the binding of LDLR to plasma PCSK9, and thereby it is possible to prevent uptake and degradation of LDLR. Accordingly, the anti-PCSK antibody may be usefully utilized to treat or prevent hypercholesterolemia, hyperlipidaemia, atherosclerotic cardiovascular disease (ACVD), acute coronary syndrome (ACS), hypertension, diabetes, stroke, Alzheimer's disease, and dyslipidemia.

### TECHNICAL FIELD

The present invention relates to the field of antibody pharmaceuticals, and specifically, the present invention relates to an antibody that specifically binds to proprotein convertase subtilisin/kexin type 9 (PCSK9), uses thereof, and a method of producing the same.

### BACKGROUND ART

Hyperlipidaemia refers to a condition in which fat constituents, such as low-density lipoprotein cholesterol (LDL), high-density lipoprotein cholesterol (HDL), ultra-low-density lipoprotein cholesterol (VLDL), and triglycerides, are present in an amount more than a necessary amount in the blood and cause inflammation. In addition, although hyperlipidaemia itself does not cause any particular symptoms, it significantly increases mortality due to cardiovascular disease as a risk factor such as hypertension, arteriosclerosis, and stroke. Patients whose lipid concentration is not controlled need to lower the risk of cardiovascular diseases by using drugs including statins to control blood lipids.

Statins, which have been used since the 1990s, are effective in controlling blood LDL cholesterol and lowering the occurrence of cardiovascular disease and mortality thereof, but have side effects such as liver toxicity and muscle toxicity. In addition, there are patients with hereditary hypercholesterolemia in which LDL cholesterol levels are not sufficiently controlled despite taking statins. It has been reported around 2003 that the proprotein convertase subtilisin/kexin type 9 (hereinafter, PCSK9) plays a key role in controlling LDL cholesterol (Maxwell, K. N. et. al. Novel putative SREBP and LXR target genes identified by microarray analysis in liver of cholesterol-fed mice. J. Lipid Res. 44, 2109-2119, 2003).

In 2006, autosomal dominant hypercholesterolemia (ADH) caused by a gene mutation was reported. Specifically, it has been reported that LDL cholesterol in the blood is severely increased when there is an abnormality in the LDLR, APOB, PCSK9, and LDLRAP1 genes. Coronary artery disease occurs when high levels of LDL cholesterol in the blood persist for a long time. It has been mainly studied in Europe and the United States, and heterozygous ADH is expected to occur in 1 in 500 and homozygous ADH is expected to occur in 1 in 1 million. It is known that ADH patients have an odds ratio of nearly ten times the incidence of coronary artery disease. ADH is an inherited disease in a family history and follows an autosomal dominant inheritance pattern. This means that if at least one parent has ADH, there is a 50% chance that the child will also have the same disease.

According to the lipid guidelines of the American College of Cardiology (ACC) and the American Heart Association (AHA) published in 2013, it has been reported that when a monoclonal antibody, a PCSK9 inhibitor, is used in patients with atherosclerotic cardiovascular disease (ASCVD), it may increase the LDL cholesterol circulation rate and increase the number of LDLR. In addition, it has been reported that the group of patients who need to be prescribed PCSK9 inhibitors may include atherosclerotic cardiovascular disease (ASCVD) in which LDL cholesterol does not reach the target level, acute coronary syndrome (ACS), unplanned coronary intervention, recurrence of ischemic stroke within 5 years, hypercholesterolemia, and hypertension. In addition, it is recommended that PCSK9 inhibitors may be administered to patients who have familial hypercholesterolemia without ASCVD or do not have statin intolerance.

Therefore, there is need for a monoclonal antibody that is a PCSK9 inhibitor, and in particular, an antibody having safety and effectiveness may be widely applied industrially.

### DETAILED DESCRIPTION OF INVENTION

### Technical Problem

The present invention relates to an anti-PCSK9 antibody that specifically binds to PCSK9. Based on the human PCSK9 protein sequence, the PCSK9 protein in which amino acids at a specific site within the range that do not affect the function were removed was expressed and purified using prokaryotic cells and used as an immunogen. This was used to prepare an anti-PCSK9 antibody that specifically binds to PCSK9. The prepared antibody may increase LDL uptake into cells by inhibiting the binding of LDLR to PCSK9.

### Solution to Problem

In order to achieve the above object, in one aspect of the present invention, there is provided a pharmaceutical composition for treating or preventing cholesterol-related diseases, comprising an antibody that specifically binds to the 209th to 218th amino acids of human PCSK9 as an active ingredient.

In another aspect of the present invention, there is provided an antibody specific for PCSK9 comprising a light chain variable region comprising LCDR1 of SEQ ID NO: 1, LCDR2 of SEQ ID NO: 2, and LCDR3 of SEQ ID NO: 3, and a heavy chain variable region comprising HCDR1 of SEQ ID NO: 4, HCDR2 of SEQ ID NO: 5, and HCDR3 of SEQ ID NO: 6; and a pharmaceutical composition thereof.

In another aspect of the present invention, there is provided a polynucleotide encoding an antibody specific for PCSK9.

In another aspect of the present invention, there is provided a vector comprising a polynucleotide encoding an antibody specific for PCSK9.

In another aspect of the present invention, there is provided a transformed cell into which the vector is introduced.

In another aspect of the present invention, there is provided a method of producing an antibody specific for PCSK9, comprising: culturing the transformed cell into which the vector is introduced; and obtaining the antibody specific for PCSK9 from the culture solution.

In another aspect of the present invention, there is provided a use of a composition comprising an antibody that specifically binds to the 209th to 218th amino acids of human PCSK9 as an active ingredient for the treatment or prevention of cholesterol-related diseases.

In another aspect of the present invention, there is provided a method for treating or preventing cholesterol-related diseases, comprising administering to a subject a pharmaceutical composition comprising an antibody that specifically binds to the 209th to 218th amino acids of human PCSK9 as an active ingredient.

### Effects of Invention

The anti-PCSK9 antibody of the present invention specifically binds to PCSK9, but binds to an epitope different from a conventionally known epitope. In addition, the anti-PCSK9 antibody of the present invention has very good binding ability with PCSK9. Therefore, the anti-PCSK9 antibody may block the binding of LDLR to plasma PCSK9, and thus it is possible to prevent uptake and degradation of LDLR. In addition, it may increase the level and amount of LDLR expression on the cell surface and increase LDL reuptake by LDLR. Accordingly, the anti-PCSK9 antibody may be usefully utilized to treat or prevent hypercholesterolemia, hyperlipidaemia, atherosclerotic cardiovascular disease (ACVD), acute coronary syndrome (ACS), and hypertension.

### BRIEF DESCRIPTION OF DRAWINGS

FIG. 1A illustrates the DNA sequence of BST PM1 bound to the pGEX 4T-1 vector (SEQ ID NO: 58).

FIG. 1B illustrates the amino acid sequence of BST PM1 encoded by polynucleotide bound to the pGEX 4T-1 vector (SEQ ID NO: 57).

FIG. 1C illustrates the results obtained by performing the sequence alignment of the original PCSK9 and BST PM1.

FIG. 2 illustrates the results obtained by confirming the BST PM1 Ag and PCSK9 polyclonal antibody by Western blot; M: size marker, Lane 1: original PCSK9 antigen, coomassie staining, Lane 2: BST PM1 Ag, coomassie staining, Lane 3: Western blot band using the original PCSK9 antigen and PCSK9 Pab (polyclonal antibody), Lane 4: Western blot band using the BST PM1 Ag and PCSK9 Pab (polyclonal antibody).

FIG. 3A illustrates the results obtained by confirming the primary screening for selecting the monoclonal antibody by ELISA.

FIG. 3B illustrates the results obtained by confirming the secondary screening for selecting the monoclonal antibody by ELISA.

FIG. 4A illustrates the results obtained by confirming the selected eight anti-PCSK9 antibodies by FACS.

FIG. 4B illustrates the results obtained by confirming the isotypes of the selected eight anti-PCSK9 antibodies.

FIG. 5 illustrates the results obtained by confirming the purification of 9G8, 4B10, and 7D1 Mab by SDS PAGE. M: size marker, Lane 1: 9G8 Mab (monoclonal antibody). Lane 2: 4B10 Mab, Lane 3: 7D1 Mab.

FIG. 6 illustrates the results obtained by confirming the purified 9G8, 4B10, and 7D1 Mabs, and PCSK9 Ag by Western blot.

FIG. 7 illustrates the results obtained by confirming the purified 9G8, 4B10, and 7D1 Mabs, and PCSK9 Ag by ELISA.

FIG. 8 illustrates the results obtained by confirming the efficacy of the anti-PCSK9 antibody for inhibiting the binding of LDLR to PCSK9 Ag by ELISA.

FIG. 9 illustrates the results obtained by confirming the effect of the anti-PCSK9 antibody on LDL uptake by HepG2 cells.

FIG. 10 illustrates the results obtained by confirming the epitope mapping of the m7D1 anti-PCSK9 antibody.

FIG. 11 illustrates the variable region sequence of the humanized 7D1 anti-PCSK9 antibody.

FIG. 12 illustrates the results obtained by confirming the production and purification of the three IgGs of ch7D1, hz7D1.11 and hz7D1.22 by SDS-PAGE.

FIG. 13 illustrates the results obtained by analyzing the EC50 of the produced and purified ch7D1 and two hz7D1 IgGs for the antigen PCSK9 Ag.

FIGS. 14A to 14D illustrate the results obtained by analyzing the produced and purified ch7D1 and two hz7D1 IgGs for the antigen PCSK9 Ag by Octet.

### BEST MODE FOR CARRYING OUT THE INVENTION

Antibody that Specifically Binds to 209th to 218th Amino Acids of PCSK9

In one aspect of the present invention, there is provided a pharmaceutical composition for treating or preventing cholesterol-related diseases, comprising an antibody that specifically binds to the 209th to 218th amino acids of human PCSK9 as an active ingredient.

As used herein, the term “PCSK9” or “subtilisin/kexin type 9” refers to an enzyme encoded by the human PCSK9 gene on chromosome 1. PCSK9 is the ninth member of the proprotein convertase family of proteins that activate other proteins. The enzyme consists of 692 amino acid residues. It is mainly present in the liver, kidney, and small intestine, and is expressed in stromal cells, mesenchymal cells and colonic epithelial cells, and is present in the blood. In this case, the PCSK9 may have the amino acid sequence of SEQ ID NO: 49. In addition, the 209th to 218th amino acid sequence of human PCSK9 may be the amino acid sequence of SEQ ID NO: 27.

PCSK9 may mediate the degradation of LDLR (LDL receptor) present on the surface of the plasma membrane of hepatocytes. PCSK9 may bind to growth factor-like repeat homology domain-A (EGF-A) in the LDLR structure. On the other hand, the LDLR/PCSK9 complex is uptaken into hepatocytes. The uptaken LDLR/PCSK9 complex inhibits conformational change of LDLR in endosomes of hepatocytes, thereby preventing dissociation and recycling of LDLR. In addition, when the LDLR/PCSK9 complex is formed, the complex is transported into the lysosome, which degrades the protein.

In addition, the cholesterol-related disease may be any one selected from the group consisting of hypercholesterolemia, hyperlipidaemia, atherosclerotic cardiovascular disease (ACVD), acute coronary syndrome (ACS), hypertension, diabetes, stroke, Alzheimer's disease, and dyslipidemia.

In addition, “BST PM1 Ag” from which a part of PCSK9 has been removed may be used as an antigen for obtaining the antibody. As used herein, the term “BST PM1 Ag” refers to a PCSK9 protein fragment used for immunization. In addition, the antigen is also referred to as “BST PM1.” In this case, “BST PM1 Ag” is a variant of PCSK9 in which amino acids in the region corresponding to the binding site of the Amgen's patent (Korean Patent No. 10-1494932) have been removed from the PCSK9 protein within a range that does not affect the antigen activity.

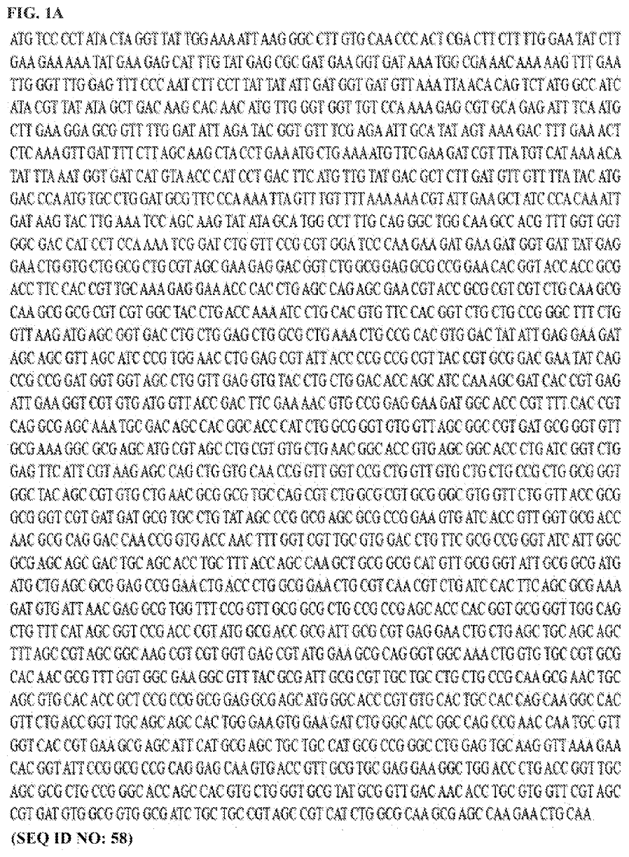

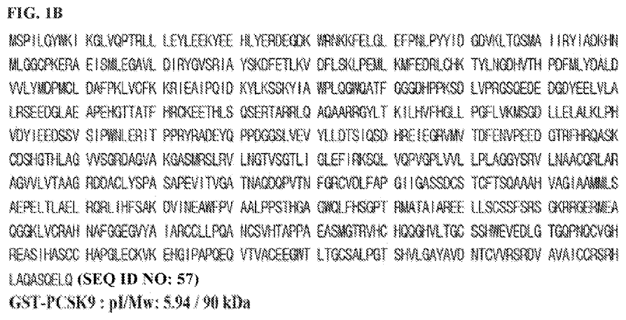

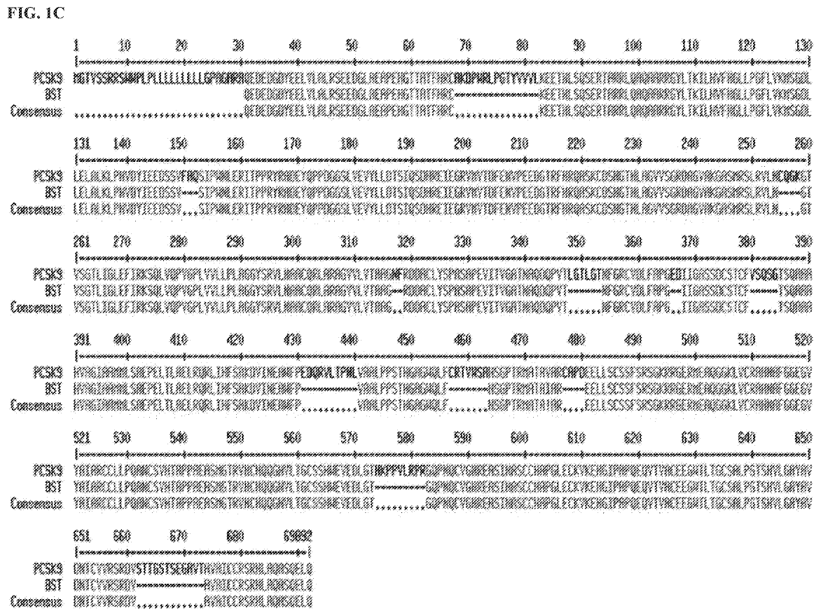

Specifically, the “BST PM1 Ag” refers to a form in which the amino acids of SEQ ID NOs: 28, 29, 30, 31, 32, 33, 34, 35, 36, 37 and 38 have been deleted from the human PCSK9 protein. This is a form in which the site where the anti-PCSK9 antibody of the Amgen's patent (regarding to anti-PCSK9 antibody) binds to the epitope in the fragment of PCSK9 has been removed. Sequence comparison of human PCSK9, BST PM1 Ag was performed by Multiple sequence alignment by Florence Corpet to compare the positions of the deleted amino acids (FIG. 1C).

Anti-PCSK9 Antibody

In another aspect of the present invention, there is provided an antibody specific for PCSK9 comprising a light chain variable region comprising LCDR1 of SEQ ID NO: 1, LCDR2 of SEQ ID NO: 2, and LCDR3 of SEQ ID NO: 3; and a heavy chain variable region comprising HCDR1 of SEQ ID NO: 4, HCDR2 of SEQ ID NO: 5, and HCDR3 of SEQ ID NO: 6; or a fragment thereof. In this case, the antibody may be a humanized antibody or a human antibody.

Specifically, the antibody may specifically bind to a polypeptide or protein having the amino acid sequence of SEQ ID NO: 27, SEQ ID NO: 49, and/or SEQ ID NO: 50 of PCSK9. Preferably, it may specifically bind to an antigen having the amino acid sequence of SEQ ID NO: 50.

In addition, the heavy chain region of the antibody may have the amino acid sequence of SEQ ID NO: 19 or SEQ ID NO; 23. In addition, the heavy chain region of the antibody may comprise or consist of an amino acid sequence having 90%, 91%, 92%, 93%, 94%, 95%, 96%, 97%, 98% or 99% identity or 100% identity to the amino acid sequence of SEQ ID NO: 19 or SEQ ID NO: 23.

In addition, the light chain region of the antibody may have the amino acid sequence of SEQ ID NO: 21 or SEQ ID NO: 25. In addition, the light chain region of the antibody may comprise or consist of an amino acid sequence having 90%, 91%, 92%, 93%, 94%, 95%, 96%, 97%, 98% or 99% identity or 100% identity to the amino acid sequence of SEQ ID NO: 21 or SEQ ID NO: 25.

As used herein, the term “antibody fragment” refers to an Fab fragment, an Fab′ fragment, or an F(ab′)₂ fragment that has an antigen-binding activity, as well as an Fv fragment that binds to human PCSK9, an scFv fragment; and includes one or more CDR regions of the antibodies described in the present invention selected from the group consisting of SEQ ID NO: 1 to SEQ ID NO: 6. The Fv fragment is the smallest antibody fragment, comprising a heavy chain variable region and a light chain variable region, without a constant region, and possessing all antigen-binding sites.

As used herein, the term “antibody” refers to an immunoglobulin, which is a structure of four peptide chains linked to each other by disulfide bonds between two identical heavy chains and two identical light chains. The different heavy chain constant regions of an immunoglobulin exhibit different amino acid compositions and sequences and thus possess different types of antigenicity. Therefore, immunoglobulins may be classified into five categories or may be referred to as immunoglobulin isotypes, i.e., IgM, IgD, IgG, IgA and IgE. The corresponding heavy chains are the μ chain, the δ chain, the γ chain, the α chain and the ε chain, respectively. The same type of Ig may be classified into different subtypes depending on the amino acid composition of the hinge region and the number and position of heavy chain disulfide bonds. For example, IgG may be classified into IgG1, IgG2, IgG3, and IgG4. Light chains may be classified into a κ or λ chain depending on different constant regions. Each of the five types of IgG may have either a κ or a λ chain. Preferably, the antibody may have a κ chain.

According to one embodiment, the anti-PCSK9 antibody of the present invention or an antigen-binding fragment thereof comprises a light chain variable region (LCVR), wherein the LCVR comprises complementarity determining regions (CDR) LCRD1, LCDR2 and LCDR3; and LCRD1, LCDR2 and LCDR3 may comprise or consist of an amino acid sequence having at least 90%, 91%, 92%, 93%, 94%, 95%, 96%, 97%, 98% or 99% identity or 100% identity to an amino acid sequence selected from the group consisting of SEQ ID NOs: 1, 2 and 3, respectively. Preferably, the LCRD1, LCDR2 and LCDR3 may have the amino acid sequence of SEQ ID NOs: 1, 2 and 3, respectively.

According to one embodiment, the anti-PCSK9 antibody of the present invention or an antigen-binding fragment thereof comprises a heavy chain variable region (HCVR), wherein the HCVR comprises complementarity determining regions (CDR) HCRD1, HCDR2 and HCDR3; and HCRD1, HCDR2 and HCDR3 may comprise or consist of an amino acid sequence having at least 90%, 91%, 92%, 93%, 94%, 95%, 96%, 97%, 98% or 99% identity or 100% identity to an amino acid sequence selected from the group consisting of SEQ ID NOs: 4, 5 and 6, respectively. Preferably, the HCRD1, HCDR2 and HCDR3 may have the amino acid sequence of SEQ ID NOs: 4, 5 and 6, respectively.

As used herein, the term “humanized antibody” refers to a chimeric antibody comprising amino acid residues from a non-human hypervariable region (HVR) and amino acid residues from a human framework (FR). In certain embodiments, a humanized antibody comprises substantially all of at least one, typically two, variable domains, wherein substantially all of the hypervariable regions (for example, CDRs) correspond to those of a non-human antibody, and substantially all of the framework regions (FRs) correspond to those of a human antibody. A humanized antibody may optionally comprise at least a portion of an antibody constant region derived from a human antibody. For example, a “humanized form” of a non-human antibody refers to an antibody that has undergone humanization.

Use of Anti-PCSK9 Antibody

In another aspect of the present invention, there is provided a pharmaceutical composition for treating or preventing cholesterol-related diseases, comprising the antibody as an active ingredient.

In this case, the cholesterol-related disease may be any one selected from the group consisting of hypercholesterolemia, hyperlipidaemia, atherosclerotic cardiovascular disease (ACVD), acute coronary syndrome (ACS), hypertension, diabetes, stroke, Alzheimer's disease, and dyslipidemia.

The pharmaceutical composition of the present invention may comprise the antibody as an active ingredient in an amount of from about 0.01% by weight to about 95% by weight, or from about 0.1% by weight to about 90% by weight, specifically from about 0.5% by weight to about 75% by weight, more specifically from about 1% by weight to about 50% by weight based on the total weight of the composition.

The pharmaceutical composition of the present invention may comprise a conventional, non-toxic pharmaceutically acceptable additive to be combined into a formulation according to a conventional method. For example, the pharmaceutical composition may further comprise a pharmaceutically acceptable carrier, diluent or excipient.

Examples of additives used in the composition of the present invention may include a sweetening agent, a binding agent, a solvent, a solubilizing agent, a wetting agent, an emulsifying agent, an isotonic agent, an absorbent, a disintegrating agent, an antioxidant, a preservative, a lubricant, a glidant, a filler, and the like.

The composition of the present invention may be prepared in a variety of formulations for parenteral administration (for example, intramuscular, intravenous or subcutaneous injection).

In addition, preparations for parenteral administration include sterile aqueous solutions, non-aqueous solutions, suspensions, emulsions, freeze-dried preparations and suppositories. As non-aqueous solvents and suspending agents, propylene glycol, polyethylene glycol, vegetable oils such as olive oil, and injectable esters such as ethyl oleate, and the like may be used. As the base of the suppositories, Witepsol, Macrogol, Tween 61, cacao butter, laurin fat, glycerogelatin, and the like may be used. On the other hand, injections may include conventional additives such as a solubilizer, an isotonic agent, a suspending agent, an emulsifying agent, a stabilizer, and a preservative.

The antibody or composition of the present invention may be administered to a patient in a therapeutically effective amount or in a pharmaceutically effective amount.

Herein, “therapeutically effective amount” or “pharmaceutically effective amount” refers to an amount of an antibody or composition effective for preventing or treating a target disease, and means an amount that is sufficient to treat a disease with a reasonable benefit/risk ratio applicable to medical treatment and does not cause side effects. The level of the effective amount may be determined depending on factors including the patient's health status, the type and severity of the disease, the activity of the drug, the sensitivity to the drug, administration method, administration time, the route of administration and excretion rate, treatment duration, the combined or concurrently used drugs, and other factors well known in the medical field.

As used herein, the term “administration” means introducing a predetermined substance to a subject by an appropriate method, and the composition may be administered through any general route as long as it may reach a target tissue. The route of administration may include intraperitoneal administration, intravenous administration, intramuscular administration, subcutaneous administration, intradermal administration, topical administration, intranasal administration, intrapulmonary administration, intrarectal administration, but is not limited thereto.

The antibody or composition of the present invention may be administered as an individual therapeutic agent or in combination with other therapeutic agents, and may be administered sequentially or simultaneously with conventional therapeutic agents, and may be administered singly or multiple times. Taking all of the above factors into consideration, it is important to administer an amount that may obtain the maximum effect with the minimum amount with minimum side effects or without side effects, which may be easily determined by those of ordinary skill in the art.

Specifically, the effective amount of the antibody in the composition of the present invention may vary depending on the age, sex, and body weight of the patient, and in general, may be administered from about 0.1 mg to about 1,000 mg, or from about 5 mg to about 200 mg per kg of body weight daily or every other day or may be divided into 1 to 3 times a day. However, since it may be increased or decreased depending on the route of administration, the severity of the disease, sex, body weight, age, and the like, the scope of the present invention is not limited thereto.

Use of Composition Comprising Anti-PCSK9 Antibody

In another aspect of the present invention, there is provided a use of a composition comprising an antibody that specifically binds to the 209th to 218th amino acids of human PCSK9 as an active ingredient for the treatment or prevention of cholesterol-related diseases. In this case, the composition and cholesterol-related disease are the same as described above.

In another aspect of the present invention, there is provided a method for treating or preventing cholesterol-related diseases, comprising administering to a subject a pharmaceutical composition comprising an antibody that specifically binds to the 209th to 218th amino acids of human PCSK9 as an active ingredient.

The subject may be effectively treated or prevented by administering the composition of the present invention to a subject having a cholesterol-related disease. In this case, the subject may be a mammal, preferably a human. In addition, the composition and types of cholesterol-related diseases are the same as described above. The route of administration, dosage, and frequency of administration of the composition may vary depending on the patient's condition and the presence or absence of side effects, and thus the composition may be administered to a subject in various ways and amounts. The optimal administration method, dosage, and frequency of administration may be selected in an appropriate range by those of ordinary skill in the art.

In another aspect of the present invention, there is provided a polynucleotide encoding an antibody specific for PCSK9. The anti-PCSK9 antibody is the same as described above. In this case, the heavy chain region of the polynucleotide may be SEQ ID NO: 20 or SEQ ID NO: 24, and the light chain region may be SEQ ID NO: 22 or SEQ ID NO: 26.

In addition, the polynucleotide may further comprise a signal sequence or a leader sequence. As used herein, the term “signal sequence” refers to a nucleic acid encoding a signal peptide that directs secretion of a target protein. The signal peptide is translated and then cleaved in a host cell. Specifically, the signal sequence of the present invention is a nucleotide encoding an amino acid sequence that initiates the migration of a protein across the endoplasmic reticulum (ER) membrane.

In another aspect of the present invention, there is provided a vector comprising a polynucleotide encoding an antibody specific for PCSK9. The heavy chain region of the polynucleotide may comprise SEQ ID NO: 20 or SEQ ID NO: 24, and the light chain region may comprise SEQ ID NO: 22 or SEQ ID NO: 26. In one embodiment, it may be a polynucleotide comprising SEQ ID NO: 20 and SEQ ID NO: 22. In one embodiment, it may be a polynucleotide comprising SEQ ID NO: 24 and SEQ ID NO: 26. In addition, the polynucleotide may further comprise a signal sequence or a leader sequence. Herein, the signal sequence is the same as described above.

In this case, the vector may be two vectors containing the combination of the polynucleotides of the heavy chain and the light chain, respectively, or a bicistronic vector containing both the combinations of the polynucleotides.

As used herein, the term “vector” may be introduced into a host cell to be recombined with and inserted into the genome of the host cell. Alternatively, the vector is understood to be a nucleic acid means containing a nucleotide sequence which is autonomously replicable as an episome. In this case, the vector may be operably linked to an appropriate promoter so that the polynucleotide may be expressed in a host cell, and the vectors include linear nucleic acids, plasmids, phagemids, cosmids, RNA vectors, viral vectors, mini-chromosomes, and analogs thereof. Examples of the viral vector include retroviruses, adenoviruses, and adeno-associated viruses, but are not limited thereto. In addition, the plasmid may contain a selectable marker such as an antibiotic resistance gene, and host cells maintaining the plasmid may be cultured under selective conditions.

As used herein, the term “gene expression” or “expression” of a target protein is understood to mean transcription of DNA sequences, translation of mRNA transcripts, and secretion of fusion protein products or fragments thereof.

In another aspect of the present invention, there is provided a transformed cell into which the vector is introduced.

As used herein, the term “transformed cell” refers to prokaryotic cells and eukaryotic cells into which a recombinant expression vector may be introduced. The transformed cell may be prepared by introducing a vector into a host cell and transforming it. In addition, the fusion protein of the present invention may be produced by expressing the polynucleotide contained in the vector.

The transformation may be performed by various methods. As long as it may produce the fusion protein of the present invention, it is not particularly limited thereto. Specifically, as the transformation method, CaCl₂) precipitation method, Hanahan method whose efficiency has been increased by using a reducing agent such as dimethyl sulfoxide (DMSO) in CaCl₂) precipitation method, electroporation, calcium phosphate precipitation method, protoplast fusion method, agitation method using silicon carbide fiber, agrobacteria mediated transformation method, transformation method using PEG, dextran sulfate, lipofectamine and dry/inhibition mediated transformation method, or the like may be used. In addition, by using the infection as a means, a target object may be delivered into a cell using virus particles. In addition, the vector may be introduced into the host cell by gene bombardment or the like.

In addition, as long as the host cell used for the production of the transformed cell may also produce the fusion protein of the present invention, it is not particularly limited thereto. Specifically, the host cells may include prokaryotic cells, eukaryotic cells, and cells of mammalian, plant, insect, fungal, or bacterial origin, but are not limited thereto. As an example of the prokaryotic cells, E. coli may be used. In addition, as an example of the eukaryotic cells, yeast may be used. In addition, as the mammalian cells, CHO cells, F2N cells, COS cells, BHK cells, Bowes melanoma cells, HeLa cells, 911 cells, AT1080 cells, A549 cells, SP2/0 cells, human lymphoblastoid, NSO cells, HT-1080 cells, PERC.6 cells, HEK 293 cells, HEK293T cells, or the like may be used, but are not limited thereto, and any cells which are known to those of ordinary skill in the art to be usable as mammalian host cells may be used.

In another aspect of the present invention, there is provided a method of producing an antibody specific for PCSK9, comprising: culturing the transformed cell into which the vector is introduced; and obtaining the antibody specific for PCSK9 from the culture solution.

As used herein, the term “culture” refers to a method of growing microorganisms in an appropriately artificially controlled environmental condition.

The method of culturing the transformed cells may be carried out using a method well known in the art. Specifically, the culture is not particularly limited as long as it may express and produce the fusion protein of the present invention. Specifically, the culture may be carried out in a batch process, or carried out continuously in a fed batch or repeated fed batch process.

In addition, the step of recovering the fusion protein from the culture may be carried out by a method known in the art. Specifically, the recovering method is not particularly limited as long as it may recover the produced fusion protein of the present invention. Preferably, the recovering method may be centrifugation, filtration, extraction, spraying, drying, evaporation, precipitation, crystallization, electrophoresis, fractional dissolution (for example, ammonium sulfate precipitation), chromatography (for example, ion exchange, affinity, hydrophobicity and size exclusion), and the like.

The antigen of the present invention was prepared by synthesizing DNA encoding BST PM1 Ag and then cloning into the expression vector pGEX 4T1. In addition, E. coli was transformed with the vector, and then IPTG was added to induce the expression of BST PM1 Ag. BST PM1 Ag expressed in a form fused with GST tag was first purified by GST affinity chromatography. Thereafter, the target protein was secondarily eluted by ion chromatography (diethylaminoethyl cellulose, DEAE). Finally, size exclusion chromatography (SEC) was performed to prepare a final protein to be used for immunization.

For the production of the hybridoma of the present invention, immunization was performed using a mouse immunized animal. Thereafter, hybridoma cells were prepared by fusion of the cells obtained from the animal and the NSO cells. The hybridoma cells that bind well to human PCSK9, BST PM1 Ag but do not bind to the GST protein were selectively sorted by ELISA screening using human PCSK9, BST PM1 Ag, and GST proteins. Thereafter, the selected three types of hybridoma cells were adapted to the serum-free medium condition, and then the appropriate cells were added to complete the culture. The purification of the antibody was performed using a Protein G column, and the antibody was eluted with an acidic elution solution, and then dialyzed against PBS to complete the preparation of the antibody. In order to prove the functional effect of the prepared antibody, the anti-PCSK9 antibody was selected through an experiment in which cells increase the uptake of LDL by inhibiting the binding of LDLR to PCSK9.

In the present invention, experiments were conducted using three final candidate anti-PCSK9 antibodies 9G8, 4B10, and 7D1 Mab. As a result of measuring binding ability with human PCSK9, BST PM1 Ag, 4B10 was the highest, followed by 9G8 and 7D1. However, as a result of conducting an experiment for the functional effect of the antibody, the 7D1 Mab was finally selected as it showed the highest level of inhibition of the binding of LDLR to PCSK9. The binding site of the selected anti-PCSK9 antibody, 7D1 Mab, to an epitope in the PCSK9 fragment was confirmed by using the linear epitope mapping method to determine the binding site of 7D1 Mab to the human PCSK9 antigen.

In addition, the light chain/heavy chain complementarity determining region of 7D1 Mab was identified through screening, and the sequence of the humanized variable region was identified. The monoclonal antibody that specifically binds to PCSK9 comprises a heavy chain variable region containing HCDR1, HCDR2 and HCDR3 sequences and a light chain variable region containing LCDR1, LCDR2 and LCDR3 sequences, and the LCDR1 sequence of the light chain variable region has the amino acid sequence of KSSQSLLDSDGKTYLN (SEQ ID NO: 1), the LCDR2 sequence has the amino acid sequence of LVSKLDS (SEQ ID NO: 2), and the LCDR3 sequence has the amino acid sequence of WQGTHFPQT (SEQ ID NO: 3). In addition, the HCDR1 sequence of the heavy chain variable region has the amino acid sequence of DYYMH (SEQ ID NO: 4), the HCDR2 sequence has the amino acid sequence of YIDPENGDTEYAPKFQG (SEQ ID NO: 5), and the HCDR3 sequence has the amino acid sequence of SPFTY (SEQ ID NO: 6) (Table 1).

TABLE 1

| LCDR1 | LCDR2 | LCDR3 |
|---|---|---|
| SEQ ID NO. 1 | SEQ ID NO: 2 | SEQ ID NO: 3 |
| KSSQSLLDSDGKTYLN | LVSKLDS | WQGTHFPQT |
| HCDR1 | HCDR1 | HCDR1 |
| SEQ ID NO: 4 | SEQ ID NO: 5 | SEQ ID NO: 6 |
| DYYMH | YIDPENGDTEYAPKFQG | SPFTY |

TABLE 2

| | |
|---|---|
| | Amino acid |
| PCSK9 Ag epitope sequence position | sequence |

| | | |
|---|---|---|
| SEQ ID NO: 27 | 209-218 | PEEDGTRFHR |

TABLE 3

| PCSK9 amino acid sequence | | | Amino acid |
|---|---|---|---|
| position | | | sequence |
| 1 | SEQ ID NO: 28 | 68 - 82 | AKDPWRLPGTYVVVL |
| 2 | SEQ ID NO: 29 | 150 - 152 | FAQ |
| 3 | SEQ ID NO: 30 | 255 - 258 | CQGK |
| 4 | SEQ ID NO: 31 | 317 - 318 | NF |
| 5 | SEQ ID NO: 32 | 348 - 353 | LGTLGT |
| 6 | SEQ ID NO: 33 | 380 - 384 | VSQSG |
| 7 | SEQ ID NO: 59 | 336 - 337 | ED |
| 8 | SEQ ID NO: 34 | 431 - 440 | EDQRVLTPNL |
| 9 | SEQ ID NO: 35 | 457 - 463 | CRTVWSA |
| 10 | SEQ ID NO: 36 | 477 - 480 | CAPD |
| 11 | SEQ ID NO: 37 | 574 - 582 | HKPPVLRPR |
| 12 | SEQ ID NO: 38 | 662 - 673 | STTGSTSEGAVT |

SEQ ID NO: 49: PCSK9 amino add sequence
MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAVARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV
DNTCVVRSRDVSTTGSTSEGAVTAVAICCRSRHLAQASQELQA
SEQ ID NO: 50: PCSK9 fragment
MGTVSSRRSWWPLPLLLLLLLLLGPAGARAQEDEDGDYEELVLALRSEED
GLAEAPEHGTTATFHRCKEETHLSQSERTARRLQAQAARRGYLTKILHVF
HGLLPGFLVKMSGDLLELALKLPHVDYIEEDSSVSIPWNLERITPPRYRA
DEYQPPDGGSLVEVYLLDTSIQSDHREIEGRVMVTDFENVPEEDGTRFHR
QASKCDSHGTHLAGVVSGRDAGVAKGASMRSLRVLNGTVSGTLIGLEFIR
KSQLVQPVGPLVVLLPLAGGYSRVLNAACQRLARAGVVLVTAAGRDDACL
YSPASAPEVITVGATNAQDQPVTNFGRCVDLFAPGIIGASSDCSTCFTSQ
AAAHVAGIAAMMLSAEPELTLAELRQRLIHFSAKDVINEAWFPVAALPPS
THGAGWQLFHSGPTRMATAVAREELLSCSSFSRSGKRRGERMEAQGGKLV
CRAHNAFGGEGVYAIARCCLLPQANCSVHTAPPAEASMGTRVHCHQQGHV
LTGCSSHWEVEDLGTGQPNQCVGHREASIHASCCHAPGLECKVKEHGIPA
PQEQVTVACEEGWTLTGCSALPGTSHVLGAYAVDNTCVVRSRDVAVAICC
RSRHLAQASQELQA
SEQ ID NO: 57: GST-PCSK9 amino acid sequence
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSQEDEDGDYEELVLALRSEEDGLAE
APEHGTTATFHRCKEETHLSQSERTARRLQAQAARRGYLTKILHVFHGLL
PGFLVKMSGDLLELALKLPHVDYIEEDSSVSIPWNLERITPPRYRADEYQ
PPDGGSLVEVYLLDTSIQSDHREIEGRVMVTDFENVPEEDGTRFHRQASK
CDSHGTHLAGVVSGRDAGVAKGASMRSLRVLNGTVSGTLIGLEFIRKSQL
VQPVGPLVVLLPLAGGYSRVLNAACQRLARAGVVLVTAAGRDDACLYSPA
SAPEVITVGATNAQDQPVTNFGRCVDLFAPGIIGASSDCSTCFTSQAAAH
VAGIAAMMLSAEPELTLAELRQRLIHFSAKDVINEAWFPVAALPPSTHGA
GWQLFHSGPTRMATAIAREELLSCSSFSRSGKRRGERMEAQGGKLVCRAH
NAFGGEGVYAIARCCLLPQANCSVHTAPPAEASMGTRVHCHQQGHVLTGC
SSHWEVEDLGTGQPNQCVGHREASIHASCCHAPGLECKVKEHGIPAPQEQ
VTVACEEGWTLTGCSALPGTSHVLGAYAVDNTCVVRSRDVAVAICCRSRH
LAQASQELQ
GST-PCSK9 → pl/Mw: 5.94 / 90 kDa
PCSK9 → pl/Mw: 5.89 / 62.6kDa
SEQ ID NO: 58: GST-PCSK9 DNA sequence
ggcgagcatgggcacccgtgtgcactgccaccagcaaggccacgttctga
ccggttgcagcagccactgggaagtggaagatctgggcaccggccagccg
aaccaatgcgttggtcaccgtgaagcgagcattcatgcgagctgctgcca
tgcgccgggcctggagtgcaaggttaaagaacacggtattccggcgccgc
aggagcaagtgaccgttgcgtgcgaggaaggctggaccctgaccggttgc
agcgcgctgccgggcaccagccacgtgctgggtgcgtatgcggttgacaa
cacctgcgtggttcgtagccgtgatgtggcggtggcgatctgctgccgta
gccgtcatctggcgcaagcgagccaagaactgcaa

TABLE 4

| | Amino acid sequence |
|---|---|
| IGHV1-2*02/ | QVQLVQSGAEVKKPGASVKVSCKASGYTFTGYYMHW |
| IGHJ4*03 | VRQAPGOGLEWMGWINPNSGGTNYAQKFQGRVTMTR |
| | DTSISTAYMELSRLRSDDTAVYYCARYFDYWGQGTL |
| | VTVSS (SEQ ID NO: 39) |
| IGKV2-30*01/ | DVVMTQSPLSLPVTLGQPASISCRSSQSLVYSDGNT |
| IGKJ4*2 | YLNWFQQRPGQSPRRLIYKVSNRDSGVPDRFSGSGS |
| | GTDFTLKISRVEAEDVGVYYCMQGTHWPLTFGGGTK |
| | VEIK (SEQ ID NO: 40) |

TABLE 5

| Item | Amino acid sequence |
|---|---|
| ch7D1 VH | EVKLVESGAELVRSGASVKLSCTASGFNIKDYYMHWVKQRPEQG |
| (SEQ ID NO: 15) | LEWIGYIDPENGDTEYAPKFQGKATMTADTSSNTAYLQLSSLTSE |
| | DTAVYYCRSSPFTYWGQGTLVTVSAASTKGPSVFPLAPSSKSTSG |
| | GTAALGCLVKDYFPEPVTVSWNSGALTSGVHTFPAVLQSSGLYSL |
| | SSVVTVPSSSLGTQTYICNVNHKPSNTKVDKKVEPKSCDKTHTCP |
| | PCPAPELLGGPSVFLFPPKPKDTLMISRTPEVTCVVVDVSHEDPEV |
| | KFNWYVDGVEVHNAKTKPREEQYNSTYRVVSVLTVLHQDWLN |
| | GKEYKCKVSNKALPAPIEKTISKAKGQPREPQVYTLPPSREEMTK |
| | NQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| ch7D1 VL | DVLMTQTPLTLSVTIGQPASISCKSSQSLLDSDGKTYLNWLLQRP |
| (SEQ ID NO: 17) | GQSPKRLIYLVSKLDSGVPDRFTGSGSGTDFTLKISRVEAEDLGV |
| | YYCWQGTHFPQTFGGGTKLELKRTVAAPSVFIFPPSDEQLKSGTA |
| | SVVCLLNNFYPREAKVQWKVDNALQSGNSQESVTEQDSKDSTY |
| | SLSSTLTLSKADYEKHKVYACEVTHQGLSSPVTKSFNRGEC |
| hz7D1.11 VH | QVQLVQSGAEVKKPGASVKVSCKASGFNIKDYYMHWVRQAPG |
| (SEQ ID NO: 19) | QGLEWIGYIDPENGDTEYAPKFQGRATMTADTSISTAYMELSRLR |
| | SDDTAVYYCRSSPFTYWGQGTLVTVSSASTKGPSVFPLAPSSKST |
| | SGGTAALGCLVKDYFPEPVTVSWNSGALTSGVHTFPAVLQSSGLY |
| | SLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDP |
| | EVKFNWYVDGVEVHNAKTKPREEQYNSTYRVVSVLTVLHQDW |
| | LNGKEYKCKVSNKALPAPIEKTISKAKGQPREPQVYTLPPSREEM |
| | TKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| hz7D11.11 VL | DVVMTQSPLSLPVTLGQPASISCKSSQSLLDSDGKTYLNWLQQRP |
| (SEQ ID NO: 21) | GQSPRRLIYLVSKLDSGVPDRFSGSGSGTDFTLKISRVEAEDVGV |
| | YYCWQGTHFPQTFGGGTKVEIKRTVAAPSVFIFPPSDEQLKSGTA |
| | SVVCLLNNFYPREAKVQWKVDNALQSGNSQESVTEQDSKDSTY |
| | SLSSTLTLSKADYEKHKVYACEVTHQGLSSPVTKSFNRGEC |
| hz7D1.22 VH | QVQLVQSGAEVKKPGASVKVSCKASGYTFKDYYMHWVRQAPG |
| (SEQ ID NO: 23) | QGLEWMGYIDPENGDTEYAPKFQGRVTMTADTSISTAYMELSRL |
| | RSDDTAVYYCRSSPFTYWGQGTLVTVSSASTKGPSVFPLAPSSKS |
| | TSGGTAALGCLVKDYFPEPVTVSWNSGALTSGVHTFPAVLQSSGL |
| | YSLSSVVTVPSSSLGTQTYICNVNHKPSNTKVDKKVEPKSCDKTH |
| | TCPPCPAPELLGGPSVFLFPPKPKDTLMISRTPEVTCVVVDVSHED |
| | PEVKFNWYVDGVEVHNAKTKPREEQYNSTYRVVSVLTVLHQD |
| | WLNGKEYKCKVSNKALPAPIEKTISKAKGQPREPQVYTLPPSREE |
| | MTKNQVSLTCLVKGFYPSDIAVEWESNGQPENNYKTTPPVLDSD |
| | GSFFLYSKLTVDKSRWQQGNVFSCSVMHEALHNHYTQKSLSLSP |
| | GK |
| hz7D11.22 VL | DVVMTQSPLSLPVTLGQPASISCKSSQSLLDSDGKTYLNWFQQRP |
| (SEQ ID NO: 25) | GQSPRRLIYLVSKLDSGVPDRFSGSGSGTDFTLKISRVEAEDVGV |
| | YYCWQGTHFPQTFGGGTKVEIKRTVAAPSVFIFPPSDEQLKSGTA |
| | SVVCLLNNFYPREAKVQWKVDNALQSGNSQESVTEQDSKDSTY |
| | SLSSTLTLSKADYEKHKVYACEVTHQGLSSPVTKSFNRGEC |

* Under bar: CDRs defined by Kabat numbering

Hereinafter, the present invention will be described in more detail by way of the following examples. However, the following examples are only for illustrating the present invention, and the scope of the present invention is not limited to the following examples.

### MODE FOR CARRYING OUT THE INVENTION

### Example 1. Cloning of BST PM1 Ag Using Prokaryotic Cells and Protein Purification

### Example 1.1. Cloning of pGEX4T-1 Vector of PCR Product of BST PM1 Gene

In order to cleave the BST PM1 gene and pGEX4T-1 vector synthesized in Genescript, the same two restriction enzymes (BamH1 and EcoR1) were used for cleavage at 37° C. for 14 hours, respectively. The vector cleaved by the restriction enzyme and the cleaved BST PM1 gene were loaded onto 1% agarose gel, and the band was identified, and then gel extraction was carried out to prepare the vector treated by the restriction enzyme the cleaved BST PM1 gene. 2 μL of pGEX4T-1 vector, 6 μL of BST PM1, 1 μL of 10× buffer, 1 μL of T4 DNA ligase (Biofact, korea) were mixed, and ligation was carried out at 4° C. for 12 hours.

After ligation, E. coli DH5α was transformed and plated on Luria-Bertani (LB) agar medium containing ampicillin (100 μg/mL), and the colonies were selected. Thereafter, in order to confirm whether the gene was properly inserted, colonies were inoculated into LB medium, cultured, and then the cells were harvested using a centrifuge. The recombinant plasmid was isolated using HiGene plasmid MiniPrpep kit (Biofact, korea) and treated with the restriction enzymes (BamH1/EcoR1). As a result, the DNA band of the BST PM1 gene and the pGEX4T-1 vector band were identified. The original PCSK9 antigen and BST PM1 antigen had a difference in sequence, and the difference was compared by sequence alignment, and the results are shown in FIG. 1C.

### Example 1.2. Expression and Purification of Recombinant Protein

In order to confirm the expression of BST PM1, E. coli BL21(DE3) cells were transformed and then plated on Luria-Bertani (LB) agar medium containing ampicillin (100 μg/mL) to generate colonies. Thereafter, the cells were obtained from the colonies and inoculated into 50 mL of Luria-Bertani (LB) medium, and then cultured at 37° C. for 12 hours. 50 mL of the cell culture solution inoculated into 5 L of LB medium containing ampicillin (100 μg/mL) was added and cultured until the OD₆₀₀ value reaches between 0.5 and 0.6, and then IPTG (isopropyl-β-D-thio-galactoside) was added to the medium at a concentration of 0.5 mM, and then further cultured at 18° C. for 16 hours.

The expressed cells were harvested by centrifugation (6,000 rpm, 30 minutes, 4° C.), and then suspended in 50 mL of a lysis buffer (PBS (phosphate buffered saline), 0.2 mM PMSF, 0.1% Triton X-100 pH 7.4), and then sonication was carried out 15 times (60 seconds/I time). The crushed cells were separated by centrifugation (13,000 rpm, 30 minutes, 4° C.) into a water-soluble cell lysate and an insoluble cell pellet. Thereafter, the water-soluble cell lysate (50 mL) was filtered with a 0.45 μm syringe filter, and then loaded onto a 5 mL GST Trap FF affinity column (GE Healthcare) equilibrated with a lysis buffer at a rate of 1 mL/min.

In order to remove non-specifically bound proteins, a lysis buffer of 10 CV (column volume) of the column volume was flowed at a rate of 2 mL/min and washed sufficiently, and then the protein sample was eluted with an elution buffer (PBS, 10 mM glutathione, pH 7.4) with a linear gradient of 1 mL/min. In order to perform the DEAE column, it was dialyzed with a lysis buffer (20 mM sodium phosphate, pH 7.4), and then loaded onto a 1 mL HighTrap DEAE FF column (GE Healthcare) at a rate of 1 mL % min.

The target protein was identified in DEAE flow-through and again isolated by size exclusion chromatography (SEC). It was loaded onto Hiprep 26/60 Sephacryl S-200 HR (GE Healthcare) equilibrated with buffer PBS (phosphate buffered saline). pH 7.4, 137 mM NaCl, 2.7 mM KCl, 10 mM Na₂HPO₄, 1.8 mM KH₂PO₄ at a rate of 0.5 mL/min. The three-step column work was performed, and the products during the separation process were identified by FPLC chromatography, and BST PM1 Ag was identified using 12% SDS PAGE gel.

### Example 2. Western Blot Using BST PM1 Ag and PCSK9 Polyclonal Antibody

In order to confirm whether the purified BST PM1 Ag binds to the PCSK9 polyclonal antibody, 1 μg of BST PM1 Ag and 1 μg of control Ag (PCSK9 (human) recombinant protein, Abnova Cat No: H00255738-P01) were loaded onto 10% SDS PAGE and then transferred to a 0.45 μm PVDF membrane. It was transferred to PBST (pH 7.4) containing 5% skim milk, and then blocked for 2 hours at room temperature, and then washed 3 times with a washing solution (PBST), and the PCSK9 polyclonal antibody (Abnova. Cat No: PAB17045) was diluted 1:1,000, and then Western blotting was performed for 1 hour at room temperature.

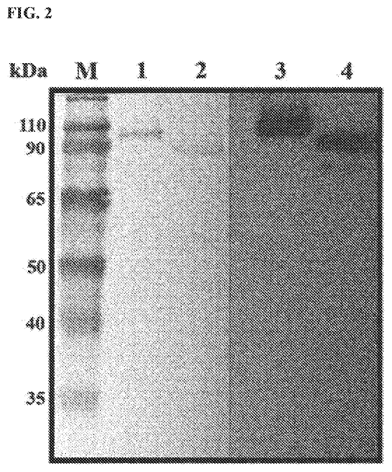

It was washed 3 times with a washing solution, and then the goat anti-rabbit IgG HRP (Santa Cruz Biotechnology, sc-2005) was diluted 1:2,500, and then incubated at room temperature for 1 hour, and washed 3 times with a washing solution, and then a band of BST PM1 Ag, PCSK9 control antigen was identified using an ECL Plus kit (FIG. 2).

### Example 3. Production of Anti-PCSK9 Mouse Antibody and Hybridoma

The BST PM1 antigen was mixed with an adjuvant (Sigma, Cat No: T2684), and the mixture was injected into the mice (BALB/c), and the blood from the mice was collected to confirm whether the antibody was produced by ELISA. After the second immunization, the antibody titer (1:5,000) was increased appropriately, and the spleen was extracted from the immunized mice, and B lymphocytes were isolated. Thereafter, it was fused with the cultured myeloma cells (sp2/0), and then the fused cells were cultured in a medium (HAT medium) containing hypoxanthine, aminopterin and thymidine, and the cells (hybridoma) in which only myeloma and B lymphocytes were fused were selectively selected and cultured.

Since B lymphocytes are normal cells, they die during long-term culture. However, myeloma cells are transformed cells, so hybridomas are selectively selected in HAT. Among the obtained hybridoma cells, cells producing an antigen-reactive antibody were identified by ELISA. The cells that were positive for ELISA were used in limiting dilution method. The process of separating positive and negative cells was repeated to produce monoclonal cells that produce an antigen-responsive antibody.

### Example 4. Screening of Anti-PCSK9 Antibody

### Example 4.1. Primary Screening

The clones that bind to BST PM1 Ag used for immunization in numerous generated monoclonal hybridoma cells and do not bind to GST tag were first selected through ELISA analysis using the following protocol. BST PM1 Ag and GST tag were diluted in 0.2 M sodium carbonate pH 9.5 in a 96-well plate (Corning Life Sciences), and added at 100 ng/well, and then incubated at 4° C. overnight to be coated. Thereafter, the plate was washed 3 times with a washing solution PBS (pH 7.4), and then the plate was incubated in 200 μL of PBST (pH 7.4) containing 5% skim milk for 1 hour at room temperature.

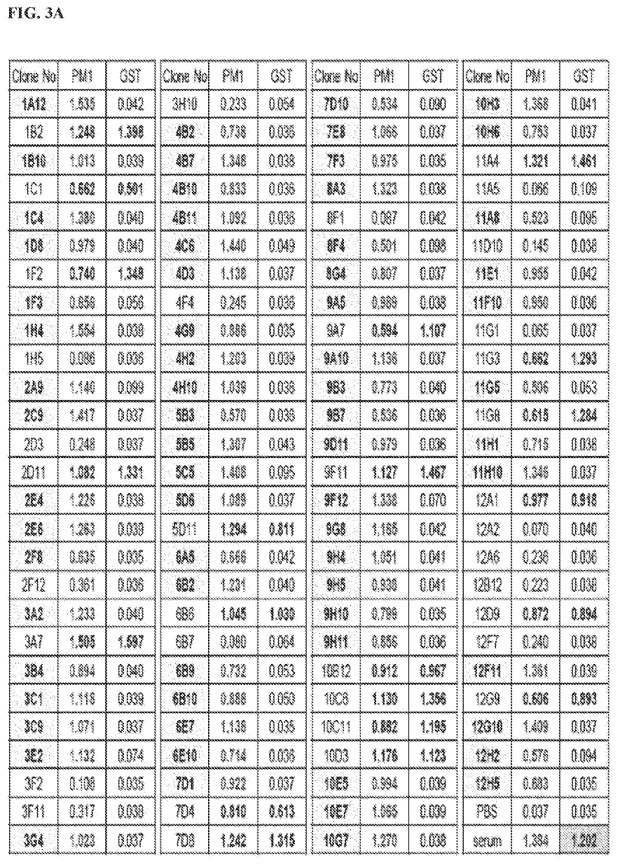

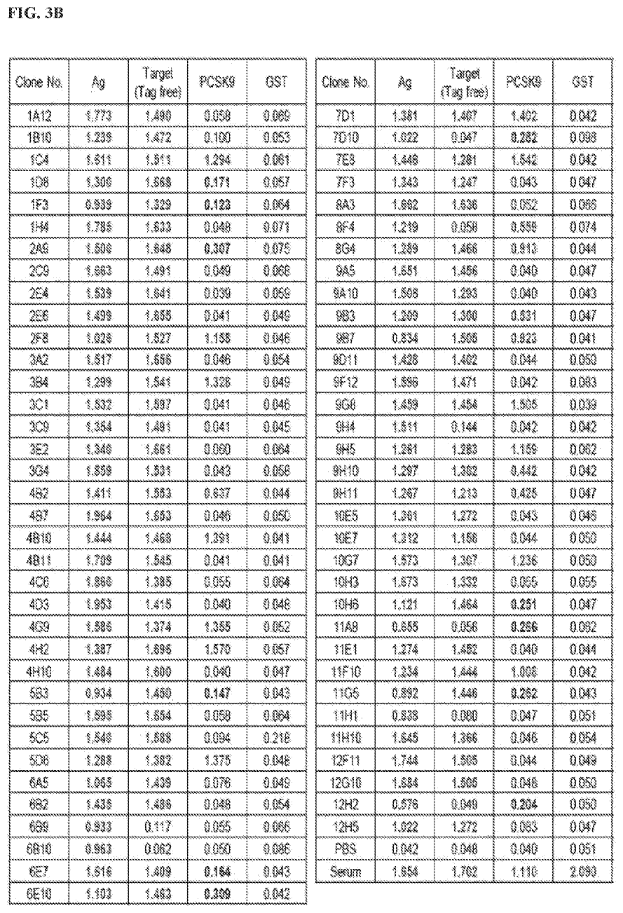

The plate was washed 3 times, and then the monoclonal hybridoma cell culture solution was added at 50 μL/well, and then it was incubated for 1 hour at room temperature and then washed 3 times. The goat anti-mouse IgG-HRP (Santa Cruz Biotechnology) at a concentration of 1:10,000 was added to the plate at 100 μL/well and incubated for 1 hour at room temperature. The plate was washed 3 times, and then the TMB solution was added at 100 μL/well and reacted for 20 minutes at room temperature, and 1 N hydrochloric acid was added at 100 μL/well and reacted for 10 minutes at room temperature. It was read immediately at 450 nm using a PerkinElmer Victor X3 plate reader. Based on the ELISA result value, 69 clones that bind to BST PM1 Ag and do not bind to GST tag were identified, and the results are shown in FIG. 3A.

### Example 4.2. Secondary Screening

The monoclonal hybridoma cells first selected through ELISA screening were diluted in 0.2 M sodium carbonate, pH 9.5 in a 96-well plate (Corning Life Sciences), and then 1) BST PM1 Ag, 2) BST PM1 GST tag removed, 3) original PCSK9, and 4) GST tag were added at 100 ng/well, and then incubated at 4° C. overnight to be coated. Thereafter, the plate was washed 3 times with a washing solution PBS (pH 7.4), and then the plate was incubated in 200 μL of PBST (pH 7.4) containing 5% skim milk for 1 hour at room temperature.

The plate was washed 3 times, and then the monoclonal hybridoma culture solution was added at 50 μL/well. It was incubated for 1 hour at room temperature and then washed 3 times. The goat anti-mouse IgG-HRP at a concentration of 1:10,000 was added to the plate at 100 μL/well and incubated for 1 hour at room temperature. The plate was washed 3 times, and then the TMB solution was added at 100 μL/well and reacted for 20 minutes at room temperature. Thereafter, 1 N hydrochloric acid was added to wells at 100 μL/well, and 10 minutes later, it was reacted at room temperature. It was read immediately at 450 nm using a PerkinElmer Victor X3 plate reader. 20 clones that do not bind to GST tag but bind to all of the remaining proteins among the 69 clones first selected were selected, and the results are shown in FIG. 3B.

### Example 4.3. Confirmation of Binding of Selected 8 Clones by Flow Cytometry (FACS)

HepG2 cells were analyzed by FACS using the culture solution of 20 hybridoma cells selected by secondary screening. HepG2 cells cultured in flask were treated with Tripsin-EDTA, and detached, and then the cells were obtained by centrifugation at 1,000 rpm for 3 minutes. The medium was removed, and then treated with PBS containing 3% FBS (3% FBS/PBS). The cells were washed, and then the cells were obtained by centrifugation at 1,000 rpm for 3 minutes. 1×10⁵ cells were treated with 1 mL of BST-PM1 hybridoma cell culture solution and then incubated on ice for 1 hour.

The cells were obtained by centrifugation at 1,000 rpm for 3 minutes, and then treated with 3% FBS/PBS, and the cells were washed, and then the cells were obtained by centrifugation at 1,000 rpm for 3 minutes. The anti-mouse IgG FITC (Invitrogen, F2761) was diluted 1.100 in 3% FBS/PBS, and then the obtained cells were treated with 1 mL of the anti-mouse IgG FITC and then incubated on ice for 1 hour. The cells were obtained by centrifugation at 1,000 rpm for 3 minutes, and then treated with 3% FBS/PBS. The cells were resuspended and then subjected to FACS analysis using a Beckman Coulter equipment (CYTOMICS FC 500).

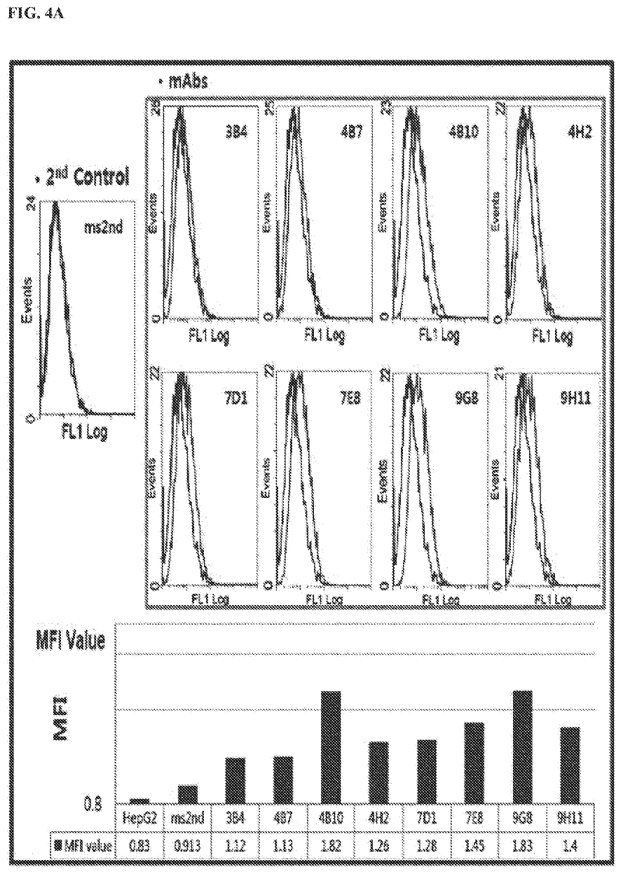

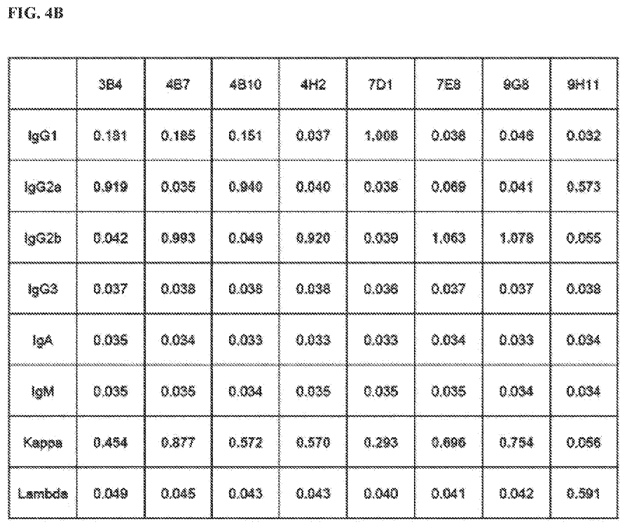

HepG2 cells and Mouse 2nd control were similarly observed with an MFI of 1.0 or less. However, as a result of analysis using the culture solution of 20 hybridomas, it was confirmed that 8 clones were bound. As a result of comparing the MFI values, the MFI values of 9G8 and 4B10 were measured to be up to 1.8, and thus the binding ability of 9G8 and 4B10 was the highest. The FACS results and MFI values are shown in FIG. 4A. In addition, the isotypes of the eight anti-PCSK9 antibodies were identified, and each type is shown in FIG. 4B.

### Example 5. Preparation of Antibodies from 9G8, 4B10, and 7D1 Hybridomas

### Example 5.1. Adaptive Culture in Serum-Free Medium

The selected three hybridoma cells (9G8, 4B10, and 7D1) were cultured in a medium containing 10% FBS. In order to grow the cells in a medium containing little FBS or without FBS, the medium adaptation was performed stepwise. Serum-free medium (SFM, Thermo fisher Cat No: 12045076) was used, and the cells were cultured to be adapted while reducing the FBS concentration stepwise. When performing each step of 1) DMEM 10% FBS medium 75%+SFM 25%, 2) DMEM 10% FBS medium 50%+SFM 50%, 3) DMEM 10% FBS medium 25%+SFM 75%, 4) DMEM 10% FBS medium 90%+SFM 10%, 4) SFM 100%, cell proliferation was performed three times to confirm growth, and then proceeded to the next step to complete cell adaptation to a serum-free medium.

### Example 5.2. Purification of 9G8, 4B10, and 7D1 Antibodies from Hybridoma

A method for purification from three hybridoma cell lines that have been adapted to a serum-free medium will be described. 2×10⁶ 9G8, 4B10, and 7D1 hybridoma cells were cultured in 10 mL of a serum-free medium in a 75T flask to proliferate the cells. The cultured cells were well detached by pipetting, and then the cells were collected by centrifugation at 1,200 rpm for 3 minutes at room temperature. Then the number of cells was measured, and the cells were put in an Erlenmeyer flask to contain 1×10⁵ cells/mL, and 100 mL of a serum-free medium was added, and the cells were cultured (1×10⁷). The cells were incubated at 37° C., 5% CO₂, 100 rpm and cultured for 8 days.

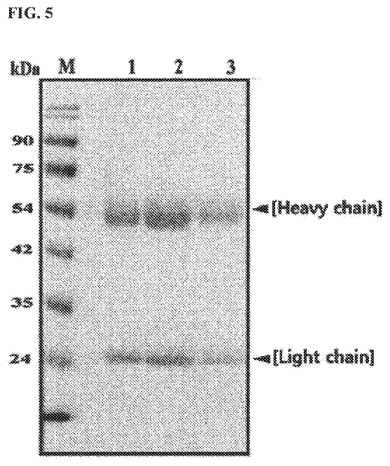

The cells were cultured for 8 days, and then the cells and lysates were removed by centrifugation at 3,000 rpm for 15 minutes at room temperature, and the supernatant was filtered with a 0.22 μm filter. A Hitrap protein G HP 5 mL (GE healthcare) was flowed sufficiently with an equilibrated solution (PBS pH 7.4), and then the filtered culture solution was loaded onto a Hitrap protein G HP 5 mL (GE healthcare) column. The non-specific binding was removed by washing with PBS sufficiently, and then the bound antibody protein was recovered using an antibody elution solution (0.1 M Glycine, pH 2.8). Immediately after recovery, 1.5 M, Tris pH 9.0 was added as much as 1/10 of the total volume to neutralize the pH. Finally, dialysis was performed with a PBS (pH 7.4) solution using a 10 kDa dialysis membrane, and the products during the purification process were identified by Protein G chromatography, and the antibody was identified using 12% SDS PAGE gel. The results are shown in FIG. 5.

### Example 6. Confirmation of Binding of 9G8, 4B10, and 7D1 Mab to PCSK9 Ag: Western Blot

In order to confirm whether the purified three types of 9G8, 4B10, and 7D1 Mab (monoclonal antibody) bind to PCSK9 Ag, western blotting was carried out. 1 μg of PCSK9 Ag (human PCSK9 protein, Acro Cat No: PC9-H5223) was loaded onto 10% SDS PAGE and then transferred to a 0.45 μm PVDF membrane. It was transferred to PBST (pH 7.4) containing 5% skim milk and then blocked for 2 hours at room temperature. It was washed 3 times with a washing solution (PBST). The three types of 9G8, 4B10, and 7D1 Mabs were quantified. 1 μg of Mabs was diluted 1:1,000 in PBST, and then incubated for 1 hour at room temperature.

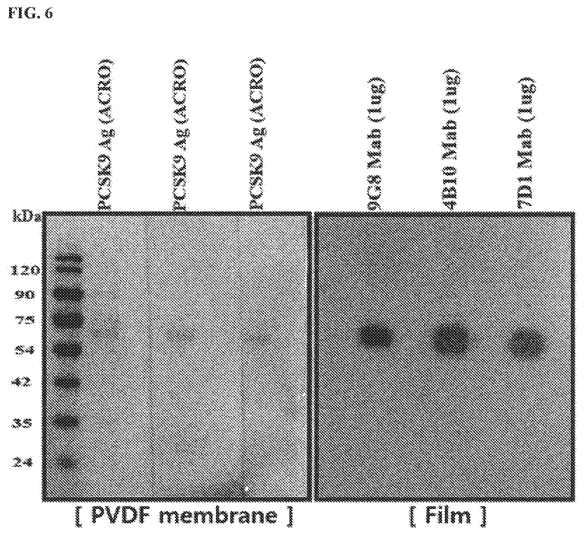

It was washed 3 times with a washing solution, and then the goat anti-mouse IgG HRP (Santa Cruz Biotechnology) was diluted 1:2.500 in PBST (pH 7.4) containing 5% skim milk. It was incubated at room temperature for 1 hour, and then washed 3 times with a washing solution. Bands of the purified three types of 9G8, 4B10, and 7D1 Mab bound to PCSK9 Ag purified as shown in FIG. 6 were identified using an ECL Plus kit. The strength of the binding ability was the highest in 4B10, followed by 9G8, and the lowest in 7D1 (FIG. 6).

### Example 7. Confirmation of Binding of 9G8, 4B10, and 7D1 Mab to PCSK9 Ag: ELISA

In order to confirm whether the purified three types of 9G8, 4B10, and 7D1 Mab bind to PCSK9 Ag, ELISA was carried out. The original PCSK9 (PCSK9 (human) recombinant protein, Abnova Cat No: H00255738-P01) was diluted to 2 μg/mL in 0.2 M sodium carbonate pH 9.5 in a 96-well plate (Corning Life Sciences). Thereafter, it was diluted by half, and then added at 100 μL/well, and then incubated at 4° C. overnight to be coated. Thereafter, the plate was washed 3 times with a washing solution PBS (pH 7.4), and then the plate was incubated in 200 μL of PBS (pH 7.4) containing 5% skim milk for 1 hour at room temperature.

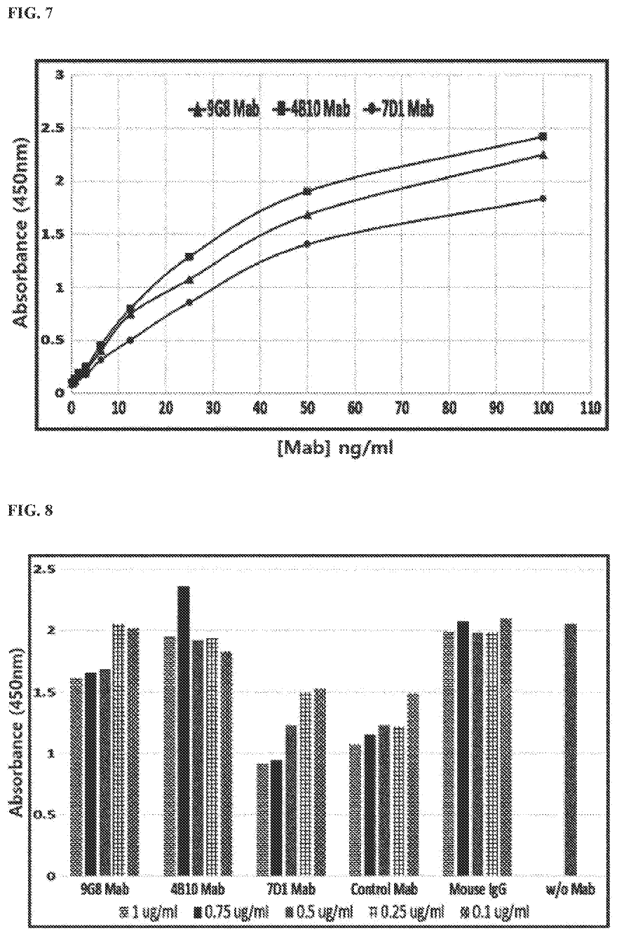

The plate was washed 3 times, and then the purified 9G8, 4B10, and 7D1 Mab were quantified, diluted to 1 μg/mL in PBS (pH 7.4), and then added to each well at 100 μL/well. It was incubated for 1 hour at room temperature and then washed 3 times with a washing solution PBS (pH 7.4). The goat anti-mouse IgG-HRP at a concentration of 1:5,000 was added to a plate at 100 μL/well and incubated for 1 hour at room temperature. The plate was washed 3 times, and then the TMB solution was added at 100 μL/well and reacted for 20 minutes at room temperature. 1 N hydrochloric acid was added at 100 μL/well, and 10 minutes later, it was reacted at room temperature. It was read immediately at 450 nm using a TECAN plate reader. As with the Western blot results of Example 6, the strength of the binding ability was the highest in 4B10, followed by 9G8, and the lowest in 7D1. The results are shown in FIG. 7.

### Example 8. Experiment of Efficacy of Anti-PCSK9 Antibody to Inhibit Binding of LDLR to PCSK9

In order to confirm the efficacy of the anti-PCSK9 antibody to block the binding of LDLR to PCSK9, ELISA was carried out. The goat anti-LDLR antibody (R&D, Cat No: AF2148) was diluted to 2 μg/mL in 0.2 M sodium carbonate pH 9.5 in a 96-well plate (Corning Life Sciences) and then added at 50 μL/well. It was incubated at 4° C. overnight to be coated. Thereafter, the plate was washed 3 times with a washing solution PBS (pH 7.4), and then the plate was incubated in 200 μL of PBS (pH 7.4) containing 2% skim milk for 1 hour at room temperature.

The plate was washed 3 times, and then LDLR (R&D, Cat No: 2148LD/CF) was diluted to 0.4 μg/mL in PBS (pH 7.4), and then added at 50 μL/well. It was incubated for 2 hours at room temperature, and the biotinylated original PCSK9 diluted to 100 ng/mL in PBS (pH 7.4), and the purified 908, 4B10, and 7D1 Mab, control Mab, and mouse IgG were diluted to 1 μg/mL in PBS (pH 7.4). Thereafter, it was diluted by half, and then was added at 50 μL/well. It was mixed and then incubated for 2 hours at room temperature. It was washed 3 times with a washing solution PBS (pH 7.4), and then the mixture (100 μL/well) was added and incubated for 1 hour at room temperature.

In order to detect the biotinylated PCSK9 bound to LDLR, streptavidin-HRP diluted to 500 ng/mL in PBS (pH 7.4) containing 1% skim milk was added at 50 μL/well, and incubated for 1 hour at room temperature. The plate was washed 3 times, and then the TMB solution was added at 100 μL/well and reacted for 20 minutes at room temperature. 1 N hydrochloric acid was added at 100 μL/well, and 10 minutes later, it was reacted at room temperature. It was read immediately at 450 nm using a TECAN plate reader.

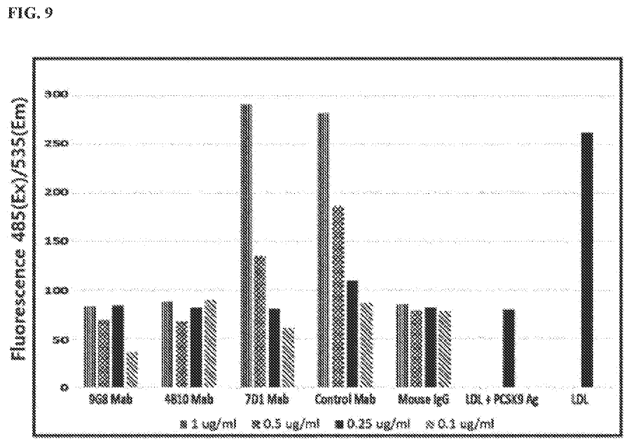

As shown in FIG. 8, it was confirmed that the 7D1 Mab inhibited the binding ability of LDLR to PCSK9 the most, which was not proportional to the binding ability of the anti-PCSK9 antibody to PCSK9. If the anti-PCSK9 antibody is functionally effective, as the amount of the antibody increases, the amount of the biotinylated-PCSK9 that binds to PCSK9 and binds to LDLR decreases, resulting in a decrease in absorbance value. It showed similar results to the control Mab (Amgen Repatha) as the control group, and it was confirmed that it was inhibited as compared to that in which Mab was not added (FIG. 8).

### Example 9. Effect of Anti-PCSK9 Antibody on LDL Uptake into HepG2 Cells

In order to confirm whether the anti-PCSK9 antibody binds to PCSK9 Ag to inhibit the decrease in LDLR and increase the LDL uptake into HepG2 cells, ELISA was carried out. HepG2 cells were added at 1×10/well using 10% FBS, DMEM medium in a 96-well plate (Costa, 3603), and incubated at 37° C., 5% CO₂ overnight. The next day, it was replaced with DMEM medium, and then incubated at 37° C., 5% CO₂ overnight.

PCSK9 Ag diluted to 2 μg/mL in DMEM medium was added at 50 μL/well, and the anti-PCSK9 antibodies (9G8, 4B10, and 7D1 Mab) were diluted to various concentrations and added at 50 μL/well. In order to produce a complex of PCSK9 Ag and anti-PCSK9 antibody, it was incubated for 1 hour at room temperature. The plate was washed 3 times with a washing solution PBS (pH 7.4). A PCSK9 Ag/anti-PCSK9 antibody mixture was added to the cells, and immediately LDL BODIPY (Invitrogen, Cat No: L3483) diluted to a final concentration of 6 μg/mL in DMEM was added at 50 μL/well. The cells were incubated at 37° C., 5% CO₂ overnight. The plate was washed 3 times, and then the cell fluorescence signal was detected at 485 (Ex)/535 (Em) using a TECAN SAFIRE™.

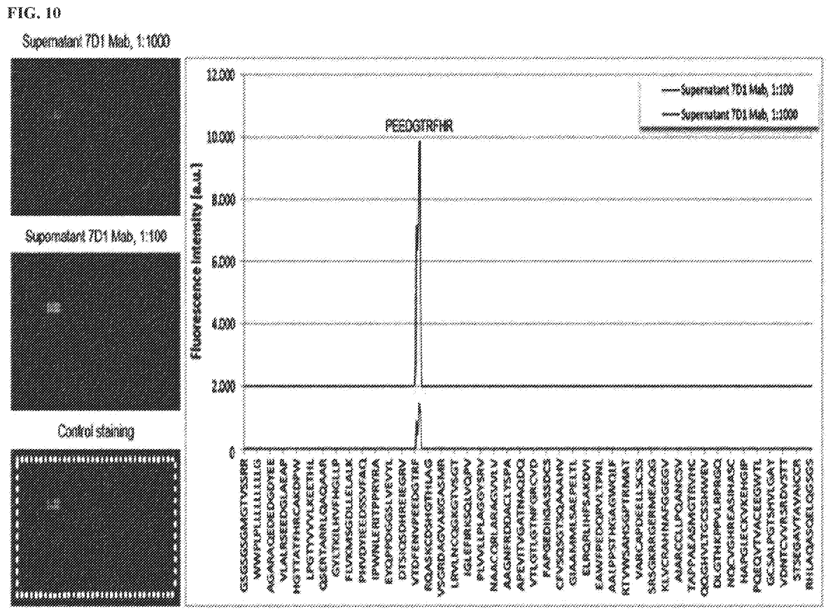

As shown in FIG. 9, LDLR expressed in HepG2 cells is not affected by PCSK9 and binds to LDL, and the fluorescence value of the uptaken LDL is about 260. In addition, when the anti-PCSK9 antibody was not added and only PCSK9 was added, the fluorescence value was about 80. As a result of comparing the anti-PCSK9 antibodies 9G8, 4B10, and 7D1 with the control Mab (Amgen Repatha), the 7D1 Mab showed the highest level of inhibiting the binding of LDLR to PCSK9, and showed similar result values to the control Mab (FIG. 9).

### Example 10. Epitope Mapping of m7D1 Anti-PCSK9 Antibody

Finally, in order to confirm the binding site of PCSK9 Ag that binds to the selected 7D1 anti-PCSK9 antibody, epitope mapping was carried out by PEPPERMAP® Epitope Substitution Scans. PCSK9 Ag composed of 695 amino acids was cleaved by 15 amino acids to make a linear peptide, and it was coated with in duplicate so that 14 amino acids overlap each other between two peptides with one amino acid spaced apart. The 7D1 hybridoma cell culture solution was diluted 1:100, 1:1,000 in the coated plate, and then incubated at 4° C. for 16 hours at 140 rpm.

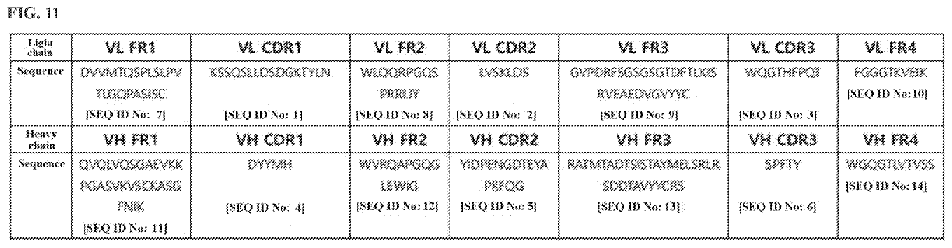

The goat anti-mouse IgG (H+L) DyLight 680 antibody (Invitrogen) was diluted to 0.2 μg/mL and then added to the plate. In order to identify the control antibody, the mouse monoclonal anti-HA DyLight 800 antibody was diluted to 0.5 μg/mL and then incubated for 45 minutes at room temperature. Detection was performed at red=700 nm/green=800 nm using the LI-COR Odyssey Imaging System. The 7D1 anti-PCSK9 antibody bound to the linear peptide of the coated PCSK9 Ag was identified, and thus the amino acid sequence of the epitope of PCSK9 Ag was identified. The experimental results are shown in FIG. 10 (FIG. 10).

### Example 11. Confirmation of Variable Region Sequence of m7D1 Anti-PCSK9 Antibody

RNA extraction (RNA prep) from the 7D1 hybridoma cell line was performed, cDNA was synthesized, and then the light chain and heavy chain variable region genes were amplified by PCR. The PCR amplified light chain and heavy chain variable genes were cloned into T-vectors. Through sequence analysis, one sequence of each of the light and heavy chains was identified, and the CDRs were indicated by the Kabat numbering method.

Fab construction cloning was performed in order to confirm whether the sequence-identified 7D1 clone binds to the antigen. The Fab was constructed by linking the variable region gene of 7D1 and the reference constant region gene identified by sequence analysis by overlapping, and then cloning to the expression vector. ELISA was performed using the periplasmic extract of the 7D1 Fab construct. The 7D1 hybridoma culture solution and the Fab have a difference in signal due to different secondary antibodies. As a positive control, the 7D1 hybridoma culture solution (the secondary antibody is anti-mouse HRP) was used. As a negative control, Fab that binds to another antigen (the secondary antibody is anti-Fab HRP) was used.

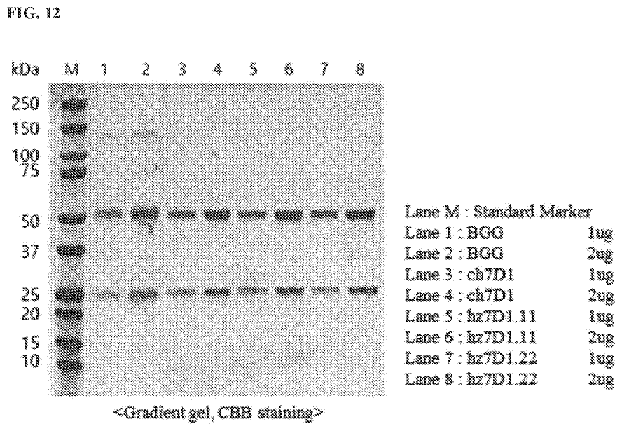

As a result of conducting an ELISA experiment using two antigens (1, original PCSK9, 2. BST PM1 Ag, 3. GST), the binding of 7D1 Fab to the positive control was confirmed for the original PCSK9 and BST PM1 Ag. The binding of PCSK9 was clear compared to the background BSA value, so the 7D1 Fab constructed as a result of hybridoma sequencing was determined to be an antibody specific for PCSK9 (FIG. 11).

### Example 12. Design of Chimeric Antibody of m7D1

In order to obtain the sequence of the human antibody required for humanization, a sequence search was conducted in IMGT (www.imgt.org), an antibody sequence database. As a result of the search, IGHV1-2*02 (75.69%) and IGKJ4*3 (79.17%) were selected for the heavy chain having the human antibody sequence most similar to the m7D1 antibody. For the light chain, IGKV2-30*01 (82.99%) and IGHJ4*02 (80.56%) were selected (Table 4). The homology was calculated based on the nucleotide sequence.

### Example 13. Humanization of Variable Region Sequence of m7D1 Anti-PCSK9 Antibody, and Production of Antibody

A humanized variable region sequence was obtained from the variable region sequence of the obtained mouse-derived m7D1 anti-PCSK9 antibody. The CDR regions in the selected human antibody sequence were substituted with the mouse 7D1 sequence to design ch7D1. Humanization was carried out by back mutation of the sequence corresponding to the framework part in the designed ch7D1 sequence into the mouse antibody sequence. In addition, two types of heavy chains (hz7D1.11 VH and hz7D1.22 VH) and two types of light chains (hz7D1.11 VL and hz7D1.22 VL) were designed with the hz7D1 sequence (Table 5).

Among the humanized versions of heavy and light chains, the 22nd version is a more human-like version, and gene synthesis was performed through GenScript so that the designed ch7D1 and two types of hz7D1 antibodies were a whole IgG form in which the heavy chain is IgG1 and the light chain is a kappa constant. The heavy and light chains of each antibody were cloned into pcDNA3.1(+) expression vector, respectively, and IgG production was performed in order to analyze the affinity of the IgG form to confirm the binding ability of the two humanized antibodies.

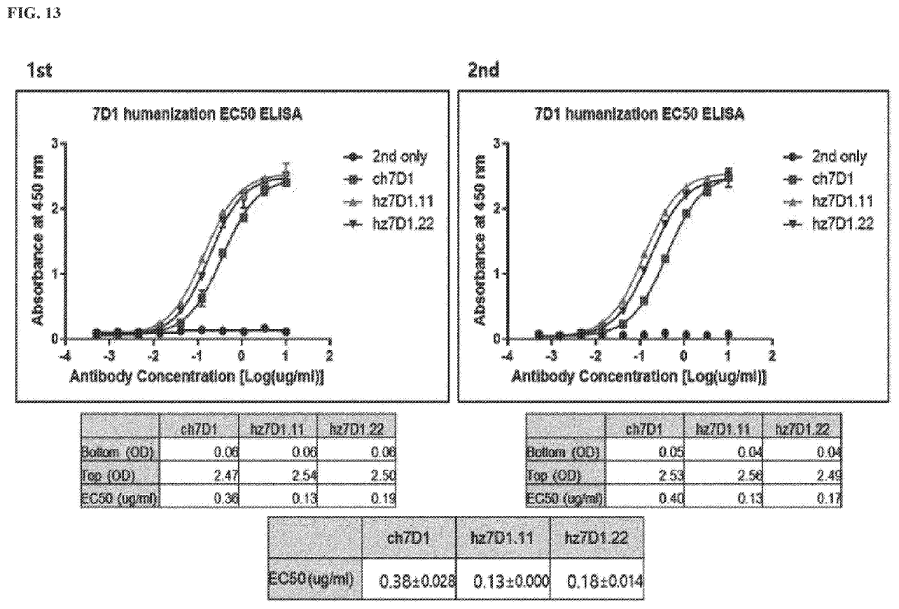

Plasmids for the heavy chain or light chain of each antibody cloned into pcDNA3.1(+) vector were identified through nucleotide sequence analysis. After transient transfection of the identified plasmid into HEK293F cells, IgG was purified from the cell culture solution through Protein A chromatography. The purity of the purified antibody was confirmed by Coomassie Blue staining after SDS PAGE (FIG. 12). As a result, it was confirmed that all three types of antibodies were purified with high purity.

### Example 14. Confirmation of Binding of Humanized 7D1 Antibody to PCSK9 Ag: ELISA

In order to confirm whether the purified three types of chimeric 7D1, hz7D1.11, and hz7D1.22 Mab bind to PCSK9 Ag, ELISA was carried out. The original PCSK9 (PCSK9 (human) recombinant protein, Abnova Cat No: H00255738-P01) was diluted to 1 μg/mL in 0.2 M sodium carbonate pH 9.5 in a 96-well plate (Corning Life Sciences), and then added at 30 μL/well, and then incubated at 4° C. overnight to be coated. Thereafter, the plate was washed 3 times with a washing solution PBS (pH 7.4), and then the plate was incubated in 200 μL of PBS (pH 7.4) containing 5% skim milk for 1 hour at room temperature.

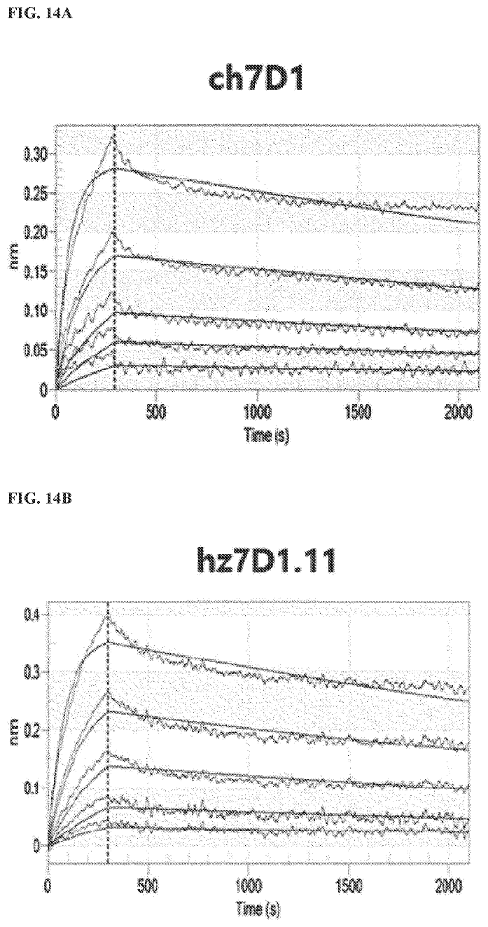

The plate was washed 3 times, and then the purified chimeric 7D1, hz7D1.11, and hz7D1.22 Mab were quantified, diluted by ⅓ starting at 10 μg/mL, and added at 30 μL/well. The plate was incubated for 1 hour at room temperature, and then washed 3 times with a washing solution PBS (pH 7.4). The anti-human IgG-HRP at a concentration of 1:3,000 was added to the plate at 30 μL/well and incubated for 1 hour at room temperature. The plate was washed 3 times. The TMB solution was added at 100 μL/well, and it was reacted for 5 minutes at room temperature. 1 N hydrochloric acid was added at 100 μL/well, and 10 minutes later, it was reacted at room temperature. Two independent tests were conducted, and the obtained results were analyzed through Prism's four parameter analysis. Based on the EC50 value, the two types of hz7D1 antibodies all showed high binding ability to the original PCSK9 antigen compared to the parental clone ch7D1 (FIG. 13).

### Example 15. Confirmation of Binding of Humanized 7D1 Antibody to PCSK9 Ag

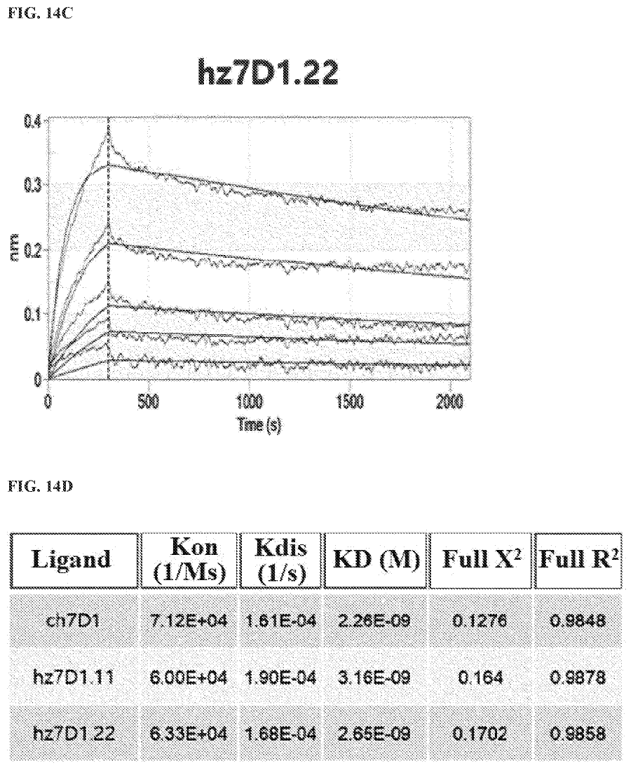

In order to confirm whether the purified three types of chimeric 7D1, hz7D1.11, and hz7D1.22 Mabs bind to PCSK9 Ag, the Octet method was carried out. The Octet test was conducted by immobilizing an IgG antibody to a sensor chip and then using antigens of various concentrations as an analyte. Immobilization was performed using CM5 chip & AR2G buffer, and the purified whole IgG 1 mg/mL was loaded. In addition, as an analyte, the original PCSK9 antigen was diluted by ½ starting at 200 nM, and loaded at 5 points. The affinity was analyzed using the 1:1 interaction model in the equipment. As a result, when the original PCSK9 was used, the two types of hz7D1 antibodies all showed the same level of binding ability as ch7D1 (FIGS. 14A to 14D).

1. A pharmaceutical composition comprising an antibody that specifically binds to the 209th to 218th amino acids of human PCSK9 as an active ingredient.

2. The pharmaceutical composition according to claim 1, wherein the PCSK9 has the amino acid sequence of SEQ ID NO: 49.

3. The pharmaceutical composition according to claim 1, wherein the 209th to 218th amino acid sequence of human PCSK9 is SEQ ID NO: 27.

4. (canceled)

5. An antibody specific for PCSK9 comprising:
a light chain variable region comprising LCDR1 of SEQ ID NO: 1, LCDR2 of SEQ ID NO: 2, and LCDR3 of SEQ ID NO: 3; and
a heavy chain variable region comprising HCDR1 of SEQ ID NO: 4, HCDR2 of SEQ ID NO: 5, and HCDR3 of SEQ ID NO: 6.

6. The antibody specific for PCSK9 according to claim 5, wherein the antibody specifically binds to an antigen having the amino acid sequence of SEQ ID NO: 27 and/or SEQ ID NO: 50 of PCSK9.

7. The antibody specific for PCSK9 according to claim 5, wherein the heavy chain region has the amino acid sequence of SEQ ID NO: 19 or SEQ ID NO: 23.

8. The antibody specific for PCSK9 according to claim 5, wherein the light chain region has the amino acid sequence of SEQ ID NO: 21 or SEQ ID NO: 25.

9. The antibody specific for PCSK9 according to claim 5, wherein the antibody is a humanized antibody.

10. A pharmaceutical composition comprising the antibody according to claim 5 as an active ingredient.

11. The pharmaceutical composition according to claim 10, wherein the pharmaceutical composition further comprises a pharmaceutically acceptable carrier.

12. (canceled)

13. A polynucleotide encoding the antibody specific for PCSK9 according to claim 5.

14. A vector comprising the polynucleotide according to claim 13.

15. A transformed cell into which the vector according to claim 14 is introduced.

16. A method of producing an antibody specific for PCSK9, comprising:
culturing the transformed cell according to claim 15; and
obtaining the antibody specific for PCSK9 from the culture solution.

17. (canceled)

18. A method for treating or preventing cholesterol-related diseases, comprising administering to a subject a pharmaceutical composition comprising an antibody that specifically binds to the 209th to 218th amino acids of human PCSK9 as an active ingredient.

19. The method according to claim 18, wherein the cholesterol-related disease is any one selected from the group consisting of hypercholesterolemia, hyperlipidaemia, atherosclerotic cardiovascular disease (ACVD), acute coronary syndrome (ACS), hypertension, diabetes, stroke, Alzheimer's disease, and dyslipidemia.

20. A method for treating or preventing cholesterol-related diseases, the method comprising administering to a subject in need thereof a therapeutically effective amount of the pharmaceutical composition according to claim 10.

21. The method according to claim 21, wherein the cholesterol-related disease is any one selected from the group consisting of hypercholesterolemia, hyperlipidaemia, atherosclerotic cardiovascular disease (ACVD), acute coronary syndrome (ACS), hypertension, diabetes, stroke, Alzheimer's disease, and dyslipidemia.
